(19) United States
(12) Patent Application Publication
(10) Pub. No.: US 2019/0055217 A1
(43) Pub. Date: Feb. 21, 2019

(54) PIPERIDINYLALKYLAMIDE DERIVATIVES HAVING MULTIMODAL ACTIVITY AGAINST PAIN

(71) Applicant: ESTEVE PHARMACEUTICALS, S.A., Barcelona (ES)

(72) Inventors: Antoni TORRENS-JOVER, Terrassa (ES); Nadine JAGEROVIC, Las Rozas (ES); Carmen ALMANSA-ROSALES, Barcelona (ES)

(21) Appl. No.: 16/090,385

(22) PCT Filed: Apr. 12, 2017
(86) PCT No.: PCT/EP2017/058748

§ 371 Date: Oct. 1, 2018

(30) Foreign Application Priority Data
Apr. 12, 2016 (EP) 16382165.5

(51) Int. Cl. C07D 401/12; C07D 417/12; C07D 413/12; A61P 25/04

(52) U.S. Cl. CPC C07D 401/12; A61P 25/04; C07D 413/12; C07D 417/12

(57) ABSTRACT

The present invention relates to piperidinylalkylamide derivatives having dual pharmacological activity towards both the sigma (σ) receptor and the μ-opioid receptor, to processes of preparation of such compounds, to pharmaceutical compositions comprising them, and to their use in therapy, in particular for the treatment of pain.

### FIELD OF THE INVENTION

The present invention relates to compounds having dual pharmacological activity towards both the sigma (σ) receptor, and the μ-opioid receptor (MOR or mu-opioid receptor) and more particularly to piperidinylalkylamide derivatives having this pharmacological activity, to processes of preparation of such compounds, to pharmaceutical compositions comprising them, and to their use in therapy, in particular for the treatment of pain.

### BACKGROUND OF THE INVENTION

The adequate management of pain constitutes an important challenge, since currently available treatments provide in many cases only modest improvements, leaving many patients unrelieved [Turk D C, Wilson H D, Cahana A. Treatment of chronic non-cancer pain. Lancet 377, 2226-2235 (2011)]. Pain affects a big portion of the population with an estimated prevalence of around 20% and its incidence, particularly in the case of chronic pain, is increasing due to the population ageing. Additionally, pain is clearly related to comorbidities, such as depression, anxiety and insomnia, which lead to important productivity losses and socio-economical burden [Goldberg D S, McGee S J. Pain as a global public health priority. BMC Public Health. 11, 770 (2011)]. Existing pain therapies include non-steroidal anti-inflammatory drugs (NSAIDs), opioid agonists, calcium channel blockers and antidepressants, but they are much less than optimal regarding their safety ratio. All of them show limited efficacy and a range of secondary effects that preclude their use, especially in chronic settings.

As mentioned before, there are few available therapeutic classes for the treatment of pain, and opioids are among the most effective, especially when addressing severe pain states. They act through three different types of opioid receptors (mu, kappa and gamma) which are transmembrane G-protein coupled receptors (GPCRs). Still, the main analgesic action is attributed to the activation of the μ-opioid receptor (MOR). However, the general administration of MOR agonists is limited due to their important side effects, such as constipation, respiratory depression, tolerance, emesis and physical dependence [Meldrum, M. L. (Ed.). Opioids and Pain Relief: A Historical Perspective. Progress in Pain Research and Management, Vol 25. IASP Press, Seattle, 2003]. Additionally, MOR agonists are not optimal for the treatment of chronic pain as indicated by the diminished effectiveness of morphine against chronic pain conditions. This is especially proven for the chronic pain conditions of neuropathic or inflammatory origin, in comparison to its high potency against acute pain. The finding that chronic pain can lead to MOR down-regulation may offer a molecular basis for the relative lack of efficacy of morphine in long-term treatment settings [Dickenson, A. H., Suzuki, R. Opioids in neuropathic pain: Clues from animal studies. Eur J Pain 9, 113-6 (2005)]. Moreover, prolonged treatment with morphine may result in tolerance to its analgesic effects, most likely due to treatment-induced MOR down-regulation, internalization and other regulatory mechanisms. As a consequence, long-term treatment can result in substantial increases in dosing in order to maintain a clinically satisfactory pain relief, but the narrow therapeutic window of MOR agonists finally results in unacceptable side effects and poor patient compliance.

The sigma-1 (σ₁) receptor was discovered 35 years ago and initially assigned to a new subtype of the opioid family, but later on and based on the studies of the enantiomers of SKF-10,047, its independent nature was established. The first link of the σ₁ receptor to analgesia was established by Chien and Pasternak [Chien C C, Pastemak G W. Sigma antagonists potentiate opioid analgesia in rats. Neurosci. Lett. 190, 137-9 (1995)], who described it as an endogenous anti-opioid system, based on the finding that σ₁ receptor agonists counteracted opioid receptor mediated analgesia, while σ₁ receptor antagonists, such as haloperidol, potentiated it.

Many additional preclinical evidences have indicated a clear role of the ao receptor in the treatment of pain [Zamanillo D, Romero L, Merlos M, Vela J M. Sigma 1 receptor A new therapeutic target for pain. Eur. J. Pharmacol, 716, 78-93 (2013)]. The development of the σ₁ receptor knockout mice, which show no obvious phenotype and perceive normally sensory stimuli, was a key milestone in this endeavour. In physiological conditions the responses of the σ₁ receptor knockout mice to mechanical and thermal stimuli were found to be undistinguishable from WT ones but they were shown to possess a much higher resistance to develop pain behaviours than WT mice when hypersensitivity entered into play. Hence, in the σ₁ receptor knockout mice capsaicin did not induce mechanical hypersensitivity, both phases of formalin-induced pain were reduced, and cold and mechanical hypersensitivity were strongly attenuated after partial sciatic nerve ligation or after treatment with paclitaxel, which are models of neuropathic pain. Many of these actions were confirmed by the use of σ₁ receptor antagonists and led to the advancement of one compound, S1RA, into clinical trials for the treatment of different pain states. Compound S1RA exerted a substantial reduction of neuropathic pain and anhedonic state following nerve injury (i.e., neuropathic pain conditions) and, as demonstrated in an operant self-administration model, the nerve-injured mice, but not sham-operated mice, acquired the operant responding to obtain it (presumably to get pain relief), indicating that σ₁ receptor antagonism relieves neuropathic pain and also address some of the comorbidities (i.e., anhedonia, a core symptom in depression) related to pain states.

Pain is multimodal in nature, since in nearly all pain states several mediators, signaling pathways and molecular mechanisms are implicated. Consequently, monomodal therapies fail to provide complete pain relief. Currently, combining existing therapies is a common clinical practice and many efforts are directed to assess the best combination of available drugs in clinical studies [Mao J, Gold M S, Backonja M. Combination drug therapy for chronic pain: a call for more clinical studies. J. Pain 12, 157-166 (2011)]. Hence, there is an urgent need for innovative therapeutics to address this unmet medical need.

As mentioned previously, opioids are among the most potent analgesics but they are also responsible for various adverse effects which seriously limit their use.

Accordingly, there is still a need to find compounds that have an alternative or improved pharmacological activity in the treatment of pain, being both effective and showing the desired selectivity, and having good “drugability” properties, i.e. good pharmaceutical properties related to administration, distribution, metabolism and excretion.

The authors of the present invention, have found a series of compounds that show dual pharmacological activity towards both the sigma (σ) receptor, and the μ-opioid receptor (MOR or mu-opioid receptor) resulting in an innovative, effective and alternative solution for the treatment of pain.

In view of the existing results of the currently available therapies and clinical practices, the present invention offers a solution by combining in a single compound binding to two different receptors relevant for the treatment of pain. This was mainly achieved by providing the compounds according to the invention that bind both to the μ-opioid receptor and to the σ₁ receptor.

### SUMMARY OF THE INVENTION

The main object of the invention is directed to piperidinylalkylamide derivatives having a dual activity binding to the σ₁ receptor and the μ-opioid receptor for use in the treatment of pain.

As this invention is aimed at providing a compound or a chemically related series of compounds which act as dual ligands of the a, receptor and the I-opioid receptor it is a very preferred embodiment if the compound has a binding expressed as Kᵢ which is preferably <1000 nM for both receptors, more preferably <500 nM, even more preferably <100 nM.

More particularly, the main aspect of the invention refers to a compound of general Formula (I),

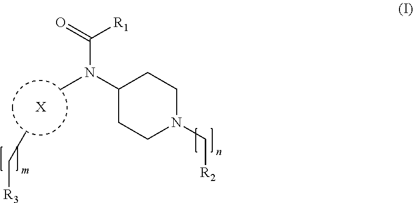

wherein R₁, R₂, R₃, m, n and X are as defined below in the detailed description.

A further object of the invention refers to the processes for preparation of compounds of general formula (I).

A still further object of the invention refers to the use of some intermediate compounds for the preparation of a compound of general formula (I).

It is also an object of the invention a pharmaceutical composition comprising a compound of formula (I).

Finally, it is an object of the invention the use of compound as a medicament and more particularly for the treatment of pain and pain related conditions.

### DETAILED DESCRIPTION OF THE INVENTION

The invention is directed to a family of structurally distinct piperidinylalkylamide derivatives which have a dual pharmacological activity towards both the sigma (σ) receptor and the μ-opioid receptor, thus solving the above problem of identifying alternative or improved pain treatments by offering such dual compounds.

The invention is directed to compounds having a dual activity binding to the σ₁ receptor and the μ-opioid receptor for use in the treatment of pain.

As this invention is aimed at providing a compound or a chemically related series of compounds which act as dual ligands of the σ₁ receptor and the μ-opioid receptor it is a preferred embodiment if the compound has a binding expressed as Kᵢ which is preferably <1000 nM for both receptors, more preferably <500 nM, even more preferably <100 nM.

The applicant has surprisingly found that the problem of providing a new effective and alternative for treating pain and pain related disorders can be solved by using a multimodal balanced analgesic approach combining two different synergistic activities in a single drug (i.e., dual ligands which are bifunctional and bind to μ-opioid receptor and to σ₁ receptor), thereby enhancing the opioid analgesia through the σ₁ activation without increasing the undesirable side effects. This supports the therapeutic value of a dual MOR/σ₁ receptor compound whereby the σ₁ receptor binding component acts as an intrinsic adjuvant of the MOR binding component.

This solution offered the advantage that the two mechanisms complement each other in order to treat pain and chronic pain using lower and better tolerated doses needed based on the potentiation of analgesia but avoiding the adverse events of μ-opioid receptor agonists.

A dual compound that possess binding to both the μ-opioid receptor and to the σ₁ receptor shows a highly valuable therapeutic potential by achieving an outstanding analgesia (enhanced in respect to the potency of the opioid component alone) with a reduced side-effect profile (safety margin increased compared to that of the opioid component alone) versus existing opioid therapies.

Advantageously, the dual compounds according to the present invention show the following functionalities: σ₁ receptor antagonism and μ-opioid receptor agonism. It has to be noted, though, that both functionalities “antagonism” and “agonism” are also sub-divided in their effect into subfunctionalities like partial agonism or inverse agonism. Accordingly, the functionalities of the dual compound should be considered within a relatively broad bandwidth.

An antagonist on one of the named receptors blocks or dampens agonist-mediated responses. Known subfunctionalities are neutral antagonists or inverse agonists.

An agonist on one of the named receptors increases the activity of the receptor above its basal level. Known subfunctionalities are full agonists, or partial agonists.

In addition, the two mechanisms complement each other since MOR agonists are only marginally effective in the treatment of neuropathic pain, while σ₁ receptor antagonists show outstanding effects in preclinical neuropathic pain models. Thus, the σ₁ receptor component adds unique analgesic actions in opioid-resistant pain. Finally, the dual approach has clear advantages over MOR agonists in the treatment of chronic pain as lower and better tolerated doses would be needed based on the potentiation of analgesia but not of the adverse events of MOR agonists.

A further advantage of using designed multiple ligands is a lower risk of drug-drug interactions compared to cocktails or multi-component drugs, thus involving simpler pharmacokinetics and less variability among patients. Additionally, this approach may improve patient compliance and broaden the therapeutic application in relation to monomechanistic drugs, by addressing more complex aetiologies. It is also seen as a way of improving the R&D output obtained using the “one drug-one target” approach, which has been questioned over the last years [Bornot A, Bauer U, Brown A, Firth M, Hellawell C, Engkvist O. Systematic Exploration of Dual-Acting Modulators from a Combined Medicinal Chemistry and Biology Perspective. J. Med. Chem, 56, 1197-1210 (2013)].

In its broader aspect, the present invention is directed to compounds of general Formula (I):

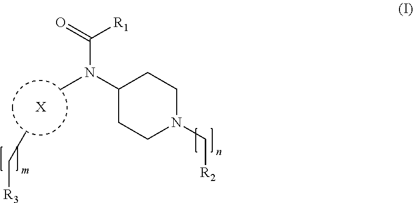

wherein

### m is O, 1, 2, 3, 4 or 5;

n is 1, 2, 3, 4 or 5;
X is a group selected from pyrimidine, pyrazine, oxadiazole, thiazole, thiadiazole, triazole and indazole;
R₁ is selected from substituted or unsubstituted C₁₋₆ alkyl, substituted or unsubstituted C₂₋₆ alkenyl, and substituted or unsubstituted C₂₋₆ alkynyl;
R₂ is selected from substituted or unsubstituted aryl and substituted or unsubstituted heterocyclyl;
R₃ is selected from substituted or unsubstituted aryl and substituted or unsubstituted heterocyclyl;

These compounds according to the invention are optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another embodiment, these compounds according to the invention are optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof.

In a further embodiment the compound according to the invention is a compound of general Formula (I′)

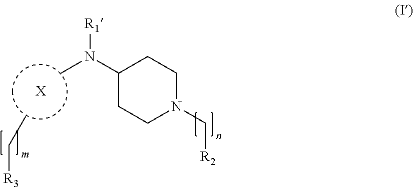

wherein, R₂, R₃, X and m are as defined in the description, and R_{1′} represents —C(O)R₄ as defined in the description or hydrogen, preferably R_{1′} is hydrogen.

In a further embodiment the compound according to the invention is a compound of general Formula (I²′)

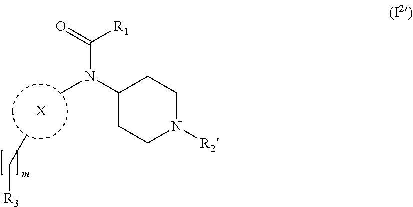

wherein, R₁, R₃, X and m are as defined in the description, and R_{2′} represents —[CH₂]ₙR₂ as defined in the description or hydrogen, preferably R_{2′} is hydrogen.

In a further embodiment the compound according to the invention is a compound of general Formula (I³′)

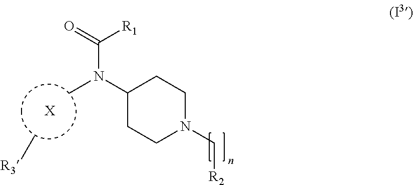

wherein, R₁, R₂, X and m are as defined in the description, and R_{3′} represents —[CH₂]ₘR₃ as defined in the description or hydrogen, preferably R_{3′} is hydrogen.

In a further embodiment the compound according to the invention is a compound of general Formula (I⁴′)

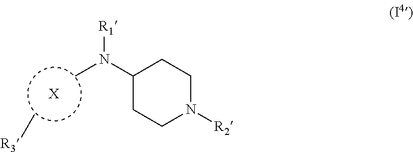

wherein X and m are as defined in the description, R_{1′} represents —C(O)R₁ as defined in the description or hydrogen, R_{2′} represents —[CH₂]ₙR₂ as defined in the description or hydrogen and R_{3′} represents —[CH₂]ₙR₃ as defined in the description or hydrogen, preferably
R_{1′} is hydrogen while R_{2′} is —[CH₂]ₙR₂ and R_{3′} is —[CH₂]ₘR₃ or
R_{2′} is hydrogen while R_{1′} is —C(O)R₁ and R_{3′} is —[CH₂]ₘR₃; or
R_{3′} is hydrogen while R_{1′} is —C(O)R₁ and R_{2′} is —[CH₂]ₙR₂; or
R_{1′} is hydrogen while R_{2′} is —[CH₂]ₙR₂ and R_{3′} is —[CH₂]ₘR₃; or
R_{1′} and R_{2′} are both hydrogen while R_{3′} is —[CH₂]ₘR₃; or
R_{1′} and R_{3′} are both hydrogen while R_{2′} is —[CH₂]ₙR₂; or
R_{3′} and R_{2′} are both hydrogen while R_{1′} is —C(O)R₁; or
R_{1′}, R_{2′} and R_{3′} are all hydrogen.

In a further embodiment the compound according to the invention is a compound of general Formula (I⁵′)

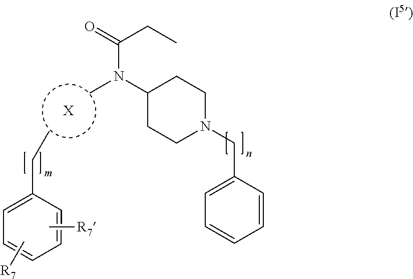

wherein X, m, n, are as defined in the description and R₇ and R_{7′} are independently selected from hydrogen, halogen, —R₆, —OR₆, —NO₂, —NR₆R_{6′″}, NR₆C(O)R_{6′}, —NR₆S(O)₂R_{6′}, —S(O)₂NR₆R_{6′}, —NR₆C(O)NR_{6′}R_{6″}, —SR₆, —S(O)R₆, S(O)₂R₆, —CN, haloalkyl, haloalkoxy, —C(O)OR₆, —C(O)NR₆R_{6′}, —OCH₂CH₂OH, —NR₆S(O)₂NR_{6′}R_{6″} and —C(CH₃)₂OR₆,
and wherein R₆, R_{6′} and R_{6″} are independently selected from hydrogen, unsubstituted C₁₋₆ alkyl, unsubstituted C₂₋₆ alkenyl and unsubstituted C₂₋₆ alkynyl; and wherein R_{6′″} is selected from hydrogen, unsubstituted C₁₋₆ alkyl, unsubstituted C₂₋₆ alkenyl, unsubstituted C₂₋₆ alkynyl and -Boc.

In a further embodiment the compound according to the invention is a compound of general Formula (I⁶′)

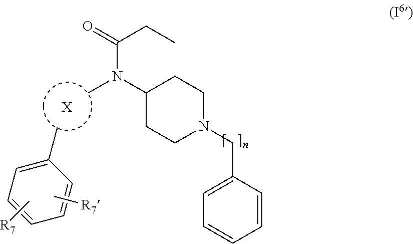

wherein X, n, R₇ and R₇′ are as defined in the description.

For clarity purposes, R₇ and R₇′ corresponds to the substitution pattern on any aryl and heterocyclyl moieties defined in R₃; and are not restricted to the phenyl moieties shown in general formulae I^{5′} and I^{6′}.

In a further embodiment the compound according to the invention is a compound of general Formula (I) having dual pharmacological activity towards both the sigma (σ) receptor and the μ-opioid receptor for use in therapy, in particular for the treatment of pain.

In a further embodiment the compound according to the invention is a compound of general Formula (I′) having dual pharmacological activity towards both the sigma (σ) receptor and the μ-opioid receptor for use in therapy, in particular for the treatment of pain.

In a further embodiment the compound according to the invention is a compound of general Formula (I²′) having dual pharmacological activity towards both the sigma (σ) receptor and the μ-opioid receptor for use in therapy, in particular for the treatment of pain.

In a further embodiment the compound according to the invention is a compound of general Formula (I³′) having dual pharmacological activity towards both the sigma (σ) receptor and the μ-opioid receptor for use in therapy, in particular for the treatment of pain.

In a further embodiment the compound according to the invention is a compound of general Formula (I⁴′) having dual pharmacological activity towards both the sigma (σ) receptor and the μ-opioid receptor for use in therapy, in particular for the treatment of pain.

In a further embodiment the compound according to the invention is a compound of general Formula (I⁵′) having dual pharmacological activity towards both the sigma (σ) receptor and the μ-opioid receptor for use in therapy, in particular for the treatment of pain.

In a further embodiment the compound according to the invention is a compound of general Formula (I⁶′) having dual pharmacological activity towards both the sigma (σ) receptor and the μ-opioid receptor for use in therapy, in particular for the treatment of pain.

For clarity purposes, all groups and definitions described in the description and referring to compounds of general Formula (I), also apply to compounds of general Formulae (I′), (I²′), (I³′) and (I⁴′) when those groups are present in the mentioned general Markush formulae.

For clarity purposes, all groups and definitions described in the description and referring to compounds of general Formula (I), also apply to compounds of general Formulae (I⁵′) and (I⁶′) when those groups are present in the mentioned general Markush formulae, since compounds of general Formula (I⁵′) and (I⁶′) are included in the general Formula (I).

In the context of this invention, alkyl is understood as meaning saturated, linear or branched hydrocarbons, which may be unsubstituted or substituted once or several times. It encompasses e.g. —CH₃ and —CH₂—CH₃. In these radicals, C₁₋₂-alkyl represents C1- or C2-alkyl, C₁₋₃-alkyl represents C1-, C2- or C3-alkyl, C₁₋₄-alkyl represents C1-, C2-, C3- or C4-alkyl, C₁₋₅-alkyl represents C1-, C2-, C3-, C4-, or C5-alkyl, C₁₋₆-alkyl represents C1-, C2-, C3-, C4-, C5- or C6-alkyl, C₁₋₇-alkyl represents C1-, C2-, C3-, C4-, C5-, C6- or C7-alkyl, C₁₋₈-alkyl represents C1-, C2-, C3-, C4-, C5-, C6-, C7- or C8-alkyl, C₁₋₁₀-alkyl represents C1-, C2-, C3-, C4-, C5-, C6-, C7-, C8-, C9- or C10-alkyl and C₁₋₁₈-alkyl represents C1-, C2-, C3-, C4-, C5-, C6-, C7-, C8-, C9-, C10-, C11-, C12-, C13-, C14-, C15-, C16-, C17- or C18-alkyl. The alkyl radicals are preferably methyl, ethyl, propyl, methylethyl, butyl, 1-methylpropyl, 2-methylpropyl, 1,1-dimethylethyl, pentyl, 1,1-dimethylpropyl, 1,2-dimethylpropyl, 2,2-dimethylpropyl, hexyl, 1-methylpentyl, if substituted also CHF₂, CF₃ or CH₂OH etc. Preferably alkyl is understood in the context of this invention as C₁₋₈ alkyl like methyl, ethyl, propyl, butyl, pentyl, hexyl, heptyl, or octyl; preferably is C₁₋₆ alkyl like methyl, ethyl, propyl, butyl, pentyl, or hexyl; more preferably is C₁₋₄ alkyl like methyl, ethyl, propyl or butyl.

Alkenyl is understood as meaning unsaturated, linear or branched hydrocarbons, which may be unsubstituted or substituted once or several times. It encompasses groups like e.g. —CH═CH—CH₃. The alkenyl radicals are preferably vinyl (ethenyl), allyl (2-propenyl). Preferably in the context of this invention alkenyl is C₂₋₁₀-alkenyl or C₂₋₈-alkenyl like ethylene, propylene, butylene, pentylene, hexylene, heptylene or octylene; or is C₂₋₆-alkenyl like ethylene, propylene, butylene, pentylene, or hexylene; or is C₂₋₄-alkenyl, like ethylene, propylene, or butylenes.

Alkynyl is understood as meaning unsaturated, linear or branched hydrocarbons, which may be unsubstituted or substituted once or several times. It encompasses groups like e.g. —C≡C—CH₃ (1-propinyl). Preferably alkynyl in the context of this invention is C₂₋₁₀-alkynyl or C₂₋₈-alkynyl like ethyne, propyne, butyene, pentyne, hexyne, heptyne, or octyne; or is C₂₋₆-alkynyl like ethyne, propyne, butyene, pentyne, or hexyne; or is C₂₋₄-alkynyl like ethyne, propyne, butyene, pentyne, or hexyne.

In connection with alkyl (also in alkylaryl, alkylheterocyclyl or alkylcycloalkyl), alkenyl, alkynyl and O-alkyl—unless defined otherwise—the term substituted in the context of this invention is understood as meaning replacement of at least one hydrogen radical on a carbon atom by halogen (F, Cl, Br, I), —NR_{c}R_{c′″}, —SR_{c}, —S(O)R_{c}, —S(O)₂R_{c}, —OR_{c}, —C(O)OR_{c}, —C(O)R_{c}, —CN, —C(O)NR_{c}R_{c′}, haloalkyl, haloalkoxy or —OC₁₋₆ alkyl, being R_{c} represented by R₄, (being R_{c′} represented by R_{4′}; being R_{c″} represented by R_{4″}; being R_{c′″} represented by R_{4′″}), wherein R₄, R_{4′}, R_{4″}, and R_{4′″} are as defined in the description. R₁ to R_{7′} are as defined in the description, and when different radicals R₁ to R_{7′} are present simultaneously in Formula I they may be identical or different.

Most preferably in connection with alkyl (also in alkylaryl, alkylheterocyclyl or alkylcycloalkyl), alkenyl, alkynyl or O-alkyl, substituted is understood in the context of this invention that any alkyl (also in alkylaryl, alkylheterocyclyl or alkylcycloalkyl), alkenyl, alkynyl or O-alkyl which, if substituted, is substituted with one or more of halogen (F, Cl, Br, I), —OR_{c}, —CN, —SR_{c}, —S(O)R_{c}, and —S(O)₂R_{c}, —C(O)R_{c}, haloalkyl, haloalkoxy or —OC₁₋₆alkyl, being R_{c} represented by R₄ (being R_{c′} represented by R_{4′}, being R_{c″} represented by R_{4″}; being R_{c′″} represented by R_{4′″}), where R₄, R_{4′}, R_{4″} and R_{4′″} are as defined in the description. R₁ to R_{7′} are as defined in the description, and when different radicals R₁ to R_{7′} are present simultaneously in Formula I, they may be identical or different.

More than one replacement on the same molecule and also on the same carbon atom is possible with the same or different substituents. This includes for example 3 hydrogens being replaced on the same C atom, as in the case of CF₃, or at different places of the same molecule, as in the case of e.g. —CH(OH)—CH═CH—CHCl₂.

In the context of this invention haloalkyl is understood as meaning an alkyl being substituted once or several times by a halogen (selected from F, Cl, Br, I). It encompasses e.g. —CH₂Cl, —CH₂F, —CHCl₂, —CHF₂, —CCl₃, —CF₃ and —CH₂—CHCl₂. Preferably haloalkyl is understood in the context of this invention as halogen-substituted C₁₋₄-alkyl representing halogen substituted C1-, C2-, C3- or C4-alkyl. The halogen-substituted alkyl radicals are thus preferably methyl, ethyl, propyl, and butyl. Preferred examples include —CH₂Cl, —CH₂F, —CHCl₂, —CHF₂, and —CF₃.

In the context of this invention haloalkoxy is understood as meaning an —O-alkyl being substituted once or several times by a halogen (selected from F, Cl, Br, I). It encompasses e.g. —OCH₂Cl, —OCH₂F, —OCHCl₂, —OCHF₂, —OCCl₃, —OCF₃ and —OCH—CHCl₂. Preferably haloalkyl is understood in the context of this invention as halogen-substituted —OC₁₋₄-alkyl representing halogen substituted C1-, C2-, C3- or C4-alkoxy. The halogen-substituted alkyl radicals are thus preferably O-methyl, O-ethyl, O-propyl, and O-butyl. Preferred examples include —OCH₂Cl, —OCH₂F, —OCHCl₂, —OCHF₂, and —OCF₃.

In the context of this invention cycloalkyl is understood as meaning saturated and unsaturated (but not aromatic) cyclic hydrocarbons (without a heteroatom in the ring), which can be unsubstituted or once or several times substituted. Furthermore, C₃₋₄-cycloalkyl represents C3- or C4-cycloalkyl, C₃₋₅-cycloalkyl represents C3-, C4- or C5-cycloalkyl, C₃₋₆-cycloalkyl represents C3-, C4-, C5- or C6-cycloalkyl, C₃₋₇-cycloalkyl represents C3-, C4-, C5-, C6- or C7-cycloalkyl, C₃₋₈-cycloalkyl represents C3-, C4-, C5-, C6-, C7- or C8-cycloalkyl, C₄₋₅-cycloalkyl represents C4- or C5-cycloalkyl, C₄₋₆-cycloalkyl represents C4-, C5- or C6-cycloalkyl, C₄₋₇-cycloalkyl represents C4-, C5-, C6- or C7-cycloalkyl, C₅₋₆-cycloalkyl represents C5- or C6-cycloalkyl and C₅₋₇-cycloalkyl represents C5-, C6- or C7-cycloalkyl. Examples are cyclopropyl, 2-methylcyclopropyl, cyclopropylmethyl, cyclobutyl, cyclopentyl, cyclopentylmethyl, cyclohexyl, cycloheptyl, cyclooctyl, and also adamantly. Preferably in the context of this invention cycloalkyl is C₃₋₈cycloalkyl like cyclopropyl, cyclobutyl, cyclopentyl, cyclohexyl, cycloheptyl, or cyclooctyl; or is C₃₋₇ cycloalkyl like cyclopropyl, cyclobutyl, cyclopentyl, cyclohexyl, or cycloheptyl; or is C₃₋₆ cycloalkyl like cyclopropyl, cyclobutyl, cyclopentyl or cyclohexyl, especially cyclopentyl or cyclohexyl.

Aryl is understood as meaning 5 to 18 membered mono or polycyclic ring systems with at least one aromatic ring but without heteroatoms even in only one of the rings. Examples are phenyl, naphthyl, fluoranthenyl, fluorenyl, tetralinyl, indanyl, 9H-fluorenyl or anthracenyl radicals, which can be unsubstituted or once or several times substituted. Most preferably aryl is understood in the context of this invention as phenyl, naphthyl or anthracenyl, preferably is phenyl.

A heterocycyl radical or group (also called heterocyclyl hereinafter) is understood as meaning 5 to 18 membered mono or poly heterocyclic ring systems, with at least one saturated or unsaturated ring which contains one or more heteroatoms selected from the group consisting of nitrogen, oxygen and/or sulfur in the ring. A heterocyclic group can also be substituted once or several times.

Examples include non-aromatic heterocyclyls such as tetrahydropyran, oxazepane, morpholine, piperidine, pyrrolidine as well as heteroaryls such as furan, benzofuran, thiophene, benzothiophene, pyrrole, pyridine, pyrimidine, pyrazine, quinoline, isoquinoline, phthalazine, thiazole, isothiazole, imidazole, benzothiazole, indole, benzotriazole, carbazole and quinazoline.

Subgroups inside the heterocycyls as understood herein include heteroaryls and non-aromatic heterocyclyls.
the heteroaryl (being equivalent to heteroaromatic radicals or aromatic heterocyclyls) is an aromatic 5 to 18 membered heterocyclic ring system of one or more rings of which at least one aromatic ring contains one or more heteroatoms selected from the group consisting of nitrogen, oxygen and/or sulfur in the ring; preferably is an aromatic heterocyclic ring system of one or two rings of which at least one aromatic ring contains one or more heteroatoms selected from the group consisting of nitrogen, oxygen and/or sulfur in the ring, more preferably is selected from furan, benzofuran, thiophene, benzothiophene, pyrrole, pyridine, pyrimidine, pyrazine, quinoline, isoquinoline, phthalazine, benzothiazole, indole, benzotriazole, carbazole, quinazoline, thiazole, isothiazole, imidazole, pyrazole, oxazole, thiophene and benzimidazole;
the non-aromatic heterocyclyl is a 5 to 18 membered heterocyclic ring system of one or more rings of which at least one ring—with this (or these) ring(s) then not being aromatic—contains one or more heteroatoms selected from the group consisting of nitrogen, oxygen and/or sulfur in the ring; preferably is a heterocyclic ring system of one or two rings of which one or both rings—with this one or two rings then not being aromatic—contain/s one or more heteroatoms selected from the group consisting of nitrogen, oxygen and/or sulfur in the ring, more preferably is selected from oxazepam, pyrrolidine, piperidine, piperazine, tetrahydropyran, morpholine, indoline, oxopyrrolidine, benzodioxane, oxetane, especially is benzodioxane, morpholine, tetrahydropyran, piperidine, oxopyrrolidine, oxetane and pyrrolidine.

Preferably in the context of this invention heterocyclyl is defined as a heterocyclic ring system of one or more saturated or unsaturated rings of which at least one ring contains one or more heteroatoms selected from the group consisting of nitrogen, oxygen and/or sulfur in the ring. Preferably it is a heterocyclic ring system of one or two saturated or unsaturated rings of which at least one ring contains one or more heteroatoms selected from the group consisting of nitrogen, oxygen and/or sulfur in the ring.

Preferred examples of heterocyclyls include oxetane, oxazepan, pyrrolidine, imidazole, oxadiazole, tetrazole, pyridine, pyrimidine, piperidine, piperazine, benzofuran, benzimidazole, indazole, benzodiazole, thiazole, benzothiazole, isothiazole, tetrahydropyran, morpholine, indoline, furan, triazole, isoxazole, pyrazole, thiophene, benzothiophene, pyrrole, pyrazine, pyrrolo[2,3b]pyridine, quinoline, isoquinoline, phthalazine, benzo-1,2,5-thiadiazole, indole, benzotriazole, benzoxazole oxopyrrolidine, pyrimidine, benzodioxolane, benzodioxane, carbazole and quinazoline, especially is pyridine, pyrazine, indazole, benzodioxane, thiazole, benzothiazole, morpholine, tetrahydropyran, pyrazole, imidazole, piperidine, thiophene, indole, benzimidazole, pyrrolo[2,3b]pyridine, benzoxazole, oxopyrrolidine, pyrimidine, oxazepane, oxetane and pyrrolidine.

In the context of this invention oxopyrrolidine is understood as meaning pyrrolidin-2-one.

In connection with aromatic heterocyclyls (heteroaryls), non-aromatic heterocyclyls, aryls and cycloalkyls, when a ring system falls within two or more of the above cycle definitions simultaneously, then the ring system is defined first as an aromatic heterocyclyl (heteroaryl) if at least one aromatic ring contains an heteroatom. If no aromatic ring contains a heteroatom, then the ring system is defined as a non-aromatic heterocyclyl if at least one non-aromatic ring contains a heteroatom. If no non-aromatic ring contains a heteroatom, then the ring system is defined as an aryl if it contains at least one aryl cycle. If no aryl is present, then the ring system is defined as a cycloalkyl if at least one non-aromatic cyclic hydrocarbon is present.

In the context of this invention alkylaryl is understood as meaning an aryl group (see above) being connected to another atom through a C₁₋₆-alkyl (see above) which may be branched or linear and is unsubstituted or substituted once or several times. Preferably alkylaryl is understood as meaning an aryl group (see above) being connected to another atom through 1 to 4 (—CH₂—) groups. Most preferably alkylaryl is benzyl (i.e. —CH₂-phenyl).

In the context of this invention alkylheterocyclyl is understood as meaning an heterocyclyl group being connected to another atom through a C₁₋₆-alkyl (see above) which may be branched or linear and is unsubstituted or substituted once or several times. Preferably alkylheterocyclyl is understood as meaning an heterocyclyl group (see above) being connected to another atom through 1 to 4 (—CH₂—) groups. Most preferably alkylheterocyclyl is —CH₂-pyridine.

In the context of this invention alkylcycloalkyl is understood as meaning an cycloalkyl group being connected to another atom through a C₁₋₆-alkyl (see above) which may be branched or linear and is unsubstituted or substituted once or several times. Preferably alkylcycloalkyl is understood as meaning an cycloalkyl group (see above) being connected to another atom through 1 to 4 (—CH₂—) groups. Most preferably alkylcycloalkyl is —CH₂-cyclopropyl.

Preferably, the aryl is a monocyclic aryl. More preferably the aryl is a 5, 6 or 7 membered monocyclic aryl. Even more preferably the aryl is a 5 or 6 membered monocyclic aryl.

Preferably, the heteroaryl is a monocyclic heteroaryl. More preferably the heteroaryl is a 5, 6 or 7 membered monocyclic heteroaryl. Even more preferably the heteroaryl is a 5 or 6 membered monocyclic heteroaryl.

Preferably, the non-aromatic heterocyclyl is a monocyclic non-aromatic heterocyclyl. More preferably the non-aromatic heterocyclyl is a 4, 5, 6 or 7 membered monocyclic non-aromatic heterocyclyl. Even more preferably the non-aromatic heterocyclyl is a 5 or 6 membered monocyclic non-aromatic heterocyclyl.

Preferably, the cycloalkyl is a monocyclic cycloalkyl. More preferably the cycloalkyl is a 3, 4, 5, 6, 7 or 8 membered monocyclic cycloalkyl. Even more preferably the cycloalkyl is a 3, 4, 5 or 6 membered monocyclic cycloalkyl.

In connection with aryl (including alkyl-aryl), cycloalkyl (including alkyl-cycloalkyl), or heterocycyl (including alkyl-heterocyclyl), substituted is understood—unless defined otherwise—as meaning substitution of the ring-system of the aryl or alkyl-aryl, cycloalkyl or alkyl-cycloalkyl; heterocyclyl or alkyl-heterocyclyl with one or more of halogen (F, Cl, Br, I), —R_{c}, —OR_{c}, —CN, —NO₂, —NR_{c}R_{c′″}—, —C(O)OR_{c}, NR_{c}C(O)R_{c′}, —C(O)NR_{c}R_{c′}, —NR_{c}S(O)₂R_{c′}, ═O, —OCH₂CH₂OH, —NR_{c}C(O)NR_{c′}R_{c″}, —S(O)₂NR_{c}R_{c′}, —NR_{c}S(O)₂NR_{c′}R_{c″}, haloalkyl, haloalkoxy, —SR_{c}, —S(O)R_{c}, —S(O)₂R_{c} or C(CH₃)OR_{c}; NR_{c}R_{c′″}, with R_{c}, R_{c′}, R_{c″} and R_{c′″} independently being either H or a saturated or unsaturated, linear or branched, substituted or unsubstituted C₁₋₆-alkyl; a saturated or unsaturated, linear or branched, substituted or unsubstituted C₁₋₆-alkyl; a saturated or unsaturated, linear or branched, substituted or unsubstituted —O—C₁₋₆ alkyl (alkoxy); a saturated or unsaturated, linear or branched, substituted or unsubstituted —S—C₁₋₆ alkyl; a saturated or unsaturated, linear or branched, substituted or unsubstituted —C(O)—C₁₋₆ alkyl-group; a saturated or unsaturated, linear or branched, substituted or unsubstituted —C(O)—O—C₁₋₆ alkyl-group; a substituted or unsubstituted aryl or alkyl-aryl; a substituted or unsubstituted cycloalkyl or alkyl-cycloalkyl; a substituted or unsubstituted heterocyclyl or alkyl-heterocyclyl, being R_{c} one of R₅ or R₆, (being R_{c′} one of R_{5′} or R_{6′}; being R_{c″} one of R_{5″} or R_{6″}; being R_{c′″} one of R_{5′″} or R_{6′″}), wherein R₁ to R_{7′} are as defined in the description, and wherein when different radicals R₁ to R_{7′} are present simultaneously in Formula I they may be identical or different.

Most preferably in connection with aryl (including alkyl-aryl), cycloalkyl (including alkyl-cycloalkyl), or heterocyclyl (including alkyl-heterocyclyl), substituted is understood in the context of this invention that any aryl, cycloalkyl and heterocyclyl which is substituted (also in an alyklaryl, alkylcycloalkyl or alkylheterocyclyl) with one or more of halogen (F, Cl, Br, I), —R_{c}, —OR_{c}, —CN, —NO₂, —NR_{c}R_{c′″}, —NR_{c}C(O)R_{c′}, —NR_{c}S(O)₂R_{c′}, ═O, haloalkyl, haloalkoxy, C(CH₃)OR_{c} or —OC₁₋₄ alkyl being unsubstituted or substituted with one or more of OR_{c} or halogen (F, Cl, I, Br), —CN, or —C₁₋₄alkyl being unsubstituted or substituted with one or more of OR_{c} or halogen (F, Cl, I, Br), being R_{c} one of R₅ or R₆, (being R_{c′} one of R_{5′} or R_{6′}; being R_{c″} one of R_{5″} or R_{6″}; being R_{c′″} one of R_{5′″} or R_{6′″}), wherein R₁ to R_{7′} are as defined in the description, and wherein when different radicals R₁ to R_{7′} are present simultaneously in Formula I they may be identical or different.

Moreover, in connection with cycloalkyl (including alkyl-cycloalkyl), or heterocyclyl (including alkylheterocyclyl) namely non-aromatic heterocyclyl (including non-aromatic alkyl-heterocyclyl), substituted is also understood—unless defined otherwise—as meaning substitution of the ring-system of the cycloalkyl or alkyl-cycloalkyl; non-aromatic heterocycyl or non aromatic alkyl-heterocycyl with

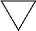

(leading to a spiro structure) or with ═O.

Moreover, in connection with cycloalkyl (including alkyl-cycloalkyl), or heterocyclyl (including alkylheterocyclyl) namely non-aromatic heterocyclyl (including non-aromatic alkyl-heterocyclyl), substituted is also understood—unless defined otherwise—as meaning substitution of the ring-system of the cycloalkyl or alkyl-cycloalkyl; non-aromatic heterocyclyl or non aromatic alkyl-heterocyclyl as spirosubstituted or substituted with ═O.

Moreover, in connection with cycloalkyl (including alkyl-cycloalkyl), or heterocyclyl (including alkylheterocyclyl) namely non-aromatic heterocyclyl (including non-aromatic alkyl-heterocyclyl), substituted is also understood—unless defined otherwise—as meaning substitution of the ring-system of the cycloalkyl or alkyl-cycloalkyl; non-aromatic heterocyclyl or non aromatic alkyl-heterocyclyl with ═O.

A ring system is a system consisting of at least one ring of connected atoms but including also systems in which two or more rings of connected atoms are joined with “joined” meaning that the respective rings are sharing one (like a spiro structure), two or more atoms being a member or members of both joined rings.

The term “leaving group” means a molecular fragment that departs with a pair of electrons in heterolytic bond cleavage. Leaving groups can be anions or neutral molecules. Common anionic leaving groups are halides such as Cl—, Br—, and I—, and sulfonate esters, such as tosylate (TsO—) or mesylate.

The term “salt” is to be understood as meaning any form of the active compound used according to the invention in which it assumes an ionic form or is charged and is coupled with a counter-ion (a cation or anion) or is in solution. By this are also to be understood complexes of the active compound with other molecules and ions, in particular complexes via ionic interactions.

The term “physiologically acceptable salt” means in the context of this invention any salt that is physiologically tolerated (most of the time meaning not being toxic—especially not caused by the counter-ion) if used appropriately for a treatment especially if used on or applied to humans and/or mammals.

These physiologically acceptable salts can be formed with cations or bases and in the context of this invention is understood as meaning salts of at least one of the compounds used according to the invention—usually a (deprotonated) acid—as an anion with at least one, preferably inorganic, cation which is physiologically tolerated—especially if used on humans and/or mammals. The salts of the alkali metals and alkaline earth metals are particularly preferred, and also those with NH₄, but in particular (mono)- or (di)sodium, (mono)- or (di)potassium, magnesium or calcium salts.

Physiologically acceptable salts can also be formed with anions or acids and in the context of this invention is understood as meaning salts of at least one of the compounds used according to the invention as the cation with at least one anion which are physiologically tolerated—especially if used on humans and/or mammals. By this is understood in particular, in the context of this invention, the salt formed with a physiologically tolerated acid, that is to say salts of the particular active compound with inorganic or organic acids which are physiologically tolerated—especially if used on humans and/or mammals. Examples of physiologically tolerated salts of particular acids are salts of: hydrochloric acid, hydrobromic acid, sulfuric acid, methanesulfonic acid, formic acid, acetic acid, oxalic acid, succinic acid, malic acid, tartaric acid, mandelic acid, fumaric acid, lactic acid or citric acid.

The compounds of the invention may be present in crystalline form or in the form of free compounds like a free base or acid.

Any compound that is a solvate of a compound according to the invention like a compound according to general formula I defined above is understood to be also covered by the scope of the invention. Methods of solvation are generally known within the art. Suitable solvates are pharmaceutically acceptable solvates. The term “solvate” according to this invention is to be understood as meaning any form of the active compound according to the invention in which this compound has attached to it via non-covalent binding another molecule (most likely a polar solvent). Especially preferred examples include hydrates and alcoholates, like methanolates or ethanolates.

Any compound that is a prodrug of a compound according to the invention like a compound according to general formula I defined above is understood to be also covered by the scope of the invention. The term “prodrug” is used in its broadest sense and encompasses those derivatives that are converted in vivo to the compounds of the invention. Such derivatives would readily occur to those skilled in the art, and include, depending on the functional groups present in the molecule and without limitation, the following derivatives of the present compounds: esters, amino acid esters, phosphate esters, metal salts sulfonate esters, carbamates, and amides. Examples of well known methods of producing a prodrug of a given acting compound are known to those skilled in the art and can be found e.g. in Krogsgaard-Larsen et al. “Textbook of Drug design and Discovery” Taylor & Francis (April 2002).

Any compound that is a N-oxide of a compound according to the invention like a compound according to general formula I defined above is understood to be also covered by the scope of the invention.

Unless otherwise stated, the compounds of the invention are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon or of a nitrogen by ¹⁵N-enriched nitrogen are within the scope of this invention.

The compounds of formula (I) as well as their salts or solvates of the compounds are preferably in pharmaceutically acceptable or substantially pure form. By pharmaceutically acceptable form is meant, inter alia, having a pharmaceutically acceptable level of purity excluding normal pharmaceutical additives such as diluents and carriers, and including no material considered toxic at normal dosage levels. Purity levels for the drug substance are preferably above 50%, more preferably above 70%, most preferably above 90%. In a preferred embodiment it is above 95% of the compound of formula (I), or of its salts. This applies also to its solvates or prodrugs.

In a more particular embodiment the compound according to the invention of general Formula (I) is a compound

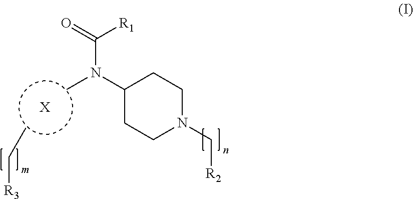

wherein
m is 0, 1, 2, 3, 4 or 5;
n is 1, 2, 3, 4 or 5;
X is a group selected from pyrimidine, pyrazine, oxadiazole, thiazole, thiadiazole, triazole and indazole;
R₁ is selected from substituted or unsubstituted C₁-6 alkyl, substituted or unsubstituted C₂₋₆ alkenyl, and substituted or unsubstituted C₂₋₆ alkynyl;
wherein the alkyl, alkenyl or alkynyl in R₁, if substituted, is substituted with one or more substituent/s selected from —OR₄, —C(O)R₄, halogen, —CN, C₁₋₄ haloalkyl, C₁₋₄ haloalkoxy and —NR₄R_{4′};
wherein R₄ is selected from hydrogen, unsubstituted C₁₋₆ alkyl, unsubstituted C₂₋₆ alkenyl and unsubstituted C₂₋₆ alkynyl;
and R_{4′} is selected from hydrogen, unsubstituted C₁₋₆ alkyl, unsubstituted C₂₋₆ alkenyl, unsubstituted C₂₋₆ alkynyl and -Boc;
   R₂ is selected from substituted or unsubstituted aryl and substituted or unsubstituted heterocyclyl;
wherein said aryl or heterocyclyl in R₂, if substituted, is substituted with one or more substituent/s selected from halogen, —R₅, —OR₅, —NO₂, —NR₅R_{5′″}—, —NR₅C(O)R_{5′}, —NR₅S(O)₂R_{5′}, —S(O)₂NR₅R_{5′}, —NR₅C(O)NR_{5′}R_{5″}, —SR₅, —S(O)R₅, S(O)₂R₅, —CN, C₁₋₄ haloalkyl, C₁₋₄ haloalkoxy, —C(O)OR₅, —C(O)NR₅R_{5′}, —OCH₂CH₂OH, —NR₅S(O)₂NR_{5′}R_{5″} and —C(CH₃)₂OR₅;
and wherein the non-aromatic heterocyclyl in R₂, if substituted, may also be spirosubstituted or substituted with ═O;
wherein R₅, R_{5′} and R_{5″} are independently selected from hydrogen, unsubstituted C₁₋₆ alkyl, unsubstituted C₂₋₆ alkenyl and unsubstituted C₂₋₆ alkynyl;
and wherein R_{5′″} is selected from hydrogen, unsubstituted C₁₋₆ alkyl, unsubstituted C₂₋₆ alkenyl, unsubstituted C₂₋₆ alkynyl and -Boc;
   R₃ is selected from substituted or unsubstituted aryl and substituted or unsubstituted heterocyclyl;
wherein said aryl or heterocyclyl in R₃, if substituted, is substituted with one or more substituent/s selected from halogen, —R₆, —OR₆, —NO₂, —NR₆R_{6′″}, NR₆C(O)R_{6′}, —NR₆S(O)₂R_{6′}, —S(O)₂NR₆R_{6′}, —NR₆C(O)NR_{6′}R_{6″}, —SR₆, —S(O)R₆, S(O)₂R₆, —CN, C₁₋₄ haloalkyl, C₁₋₄ haloalkoxy, —C(O)OR₆, —C(O)NR₆R_{6′}, —OCH₂CH₂OH, —NR₆S(O)₂NR_{6′}R_{6″} and —C(CH₃)₂OR₆;
and wherein a non-aromatic heterocyclyl in R₃, if substituted, may also be spirosubstituted or substituted with ═O;
wherein R₆, R_{6′} and R_{6″} are independently selected from hydrogen, unsubstituted C₁₋₆ alkyl, unsubstituted C₂₋₆ alkenyl and unsubstituted C₂₋₆ alkynyl;
and wherein R_{6′″} is selected from hydrogen, unsubstituted C₁₋₆ alkyl, unsubstituted C₂₋₆ alkenyl, unsubstituted C₂₋₆ alkynyl and -Boc;

These preferred compounds according to the invention are optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

m is 0, 1, 2, 3, 4 or 5;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

n is 1, 2, 3, 4 or 5;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

X is a group selected from pyrimidine, pyrazine, oxadiazole, thiazole, thiadiazole, triazole and indazole;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a preferred embodiment of the compound according to the invention of general Formula (I) is a compound wherein

X is a pyrimidine;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

X is pyrazine;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

X is oxadiazole;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

X is thiazole;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

X is thiadiazole;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

X is triazole;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

X is indazole;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

R₁ is selected from substituted or unsubstituted C₁₋₆ alkyl, substituted or unsubstituted C₂₋₆ alkenyl, and substituted or unsubstituted C₂₋₆ alkynyl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

R₁ is substituted or unsubstituted C₁₋₆ alkyl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

R₂ is selected from substituted or unsubstituted aryl and substituted or unsubstituted heterocyclyl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

R₂ is substituted or unsubstituted aryl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the according to the invention of general Formula (I) is a compound wherein

R₃ is selected from substituted or unsubstituted aryl and substituted or unsubstituted heterocyclyl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the according to the invention of general Formula (I) is a compound wherein

R₃ is substituted or unsubstituted aryl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein
R₄ is selected from hydrogen, unsubstituted C₁₋₆ alkyl, unsubstituted C₂₋₆ alkenyl and unsubstituted C₂₋₆ alkynyl;
and R_{4′} is selected from hydrogen, unsubstituted C₁₋₆ alkyl, unsubstituted C₂₋₆ alkenyl, unsubstituted C₂₋₆ alkynyl and -Boc;
   optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein
R₄ is selected from hydrogen, unsubstituted C₁₋₆ alkyl, unsubstituted C₂₋₆ alkenyl and unsubstituted C₂₋₆ alkynyl;
   optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein
R_{4′} is selected from hydrogen, unsubstituted C₁₋₆ alkyl, unsubstituted C₂₋₆ alkenyl, unsubstituted C₂₋₆ alkynyl and -Boc;
   optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

R₅, R_{5′} and R_{5″} are independently selected from hydrogen, unsubstituted C₁₋₆ alkyl, unsubstituted C₂₋₆ alkenyl and unsubstituted C₂₋₆ alkynyl;
and wherein R_{5′″} is selected from hydrogen, unsubstituted C₁₋₆ alkyl, unsubstituted C₂₋₆ alkenyl, unsubstituted C₂₋₆ alkynyl and -Boc;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

R₅, R_{5′} and R_{5″} are independently selected from hydrogen, unsubstituted C₁₋₆ alkyl, unsubstituted C₂₋₆ alkenyl and unsubstituted C₂₋₆ alkynyl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

wherein R_{5′″} is selected from hydrogen, unsubstituted C₁₋₆ alkyl, unsubstituted C₂₋₆ alkenyl, unsubstituted C₂₋₆ alkynyl and -Boc;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

R₆, R_{6′} and R_{6″} are independently selected from hydrogen, unsubstituted C₁₋₆ alkyl, unsubstituted C₂₋₆ alkenyl and unsubstituted C₂₋₆ alkynyl;
and wherein R_{6′″} is selected from hydrogen, unsubstituted C₁₋₆ alkyl, unsubstituted C₂₋₆ alkenyl, unsubstituted C₂₋₆ alkynyl and -Boc;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

R₆, R_{6′} and R_{6″} are independently selected from hydrogen, unsubstituted C₁₋₆ alkyl, unsubstituted C₂₋₆ alkenyl and unsubstituted C₂₋₆ alkynyl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

R_{6′″} is selected from hydrogen, unsubstituted C₁₋₆ alkyl, unsubstituted C₂₋₆ alkenyl, unsubstituted C₂₋₆ alkynyl and -Boc;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I^{5′}) or (I^{6′}) is a compound wherein

R₇ and R_{7′} are independently selected from halogen, —R₆, —OR₆, —NO₂, —NR₆R_{6′″}, NR₆C(O)R_{6′}, —NR₆S(O)₂R_{6′}, —S(O)₂NR₆R_{6′}, —NR₆C(O)NR_{6′}R_{6″}, —SR₆, —S(O)R₆, S(O)₂R₆, —CN, haloalkyl, haloalkoxy, —C(O)OR₆, —C(O)NR₆R_{6′}, —OCH₂CH₂OH, —NR₆S(O)₂NR_{6′}R_{6″} and —C(CH₃)₂OR₆;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I), is a compound

wherein
m is 0, 1, 2, 3, 4 or 5;
n is 1, 2, 3, 4 or 5;
X is a group selected from pyrimidine, pyrazine, oxadiazole, thiazole, thiadiazole, triazole and indazole;
R₁ is selected from substituted or unsubstituted C₁₋₆ alkyl, substituted or unsubstituted C₂₋₆ alkenyl, and substituted or unsubstituted C₂₋₆ alkynyl;
wherein
the C₁₋₆ alkyl is preferably selected from methyl, ethyl, propyl, butyl, pentyl, hexyl, isopropyl, isobutyl and 2-methylpropyl, more preferably the C₁₋₆ alkyl is methyl or ethyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;
and/or
R₂ is selected from substituted or unsubstituted aryl and substituted or unsubstituted heterocyclyl;
wherein
the aryl is selected from phenyl, naphthyl, or anthracene; preferably is naphthyl and phenyl; more preferably is phenyl;
and/or
the heterocyclyl is a heterocyclic ring system of one or more saturated or unsaturated rings of which at least one ring contains one or more heteroatoms selected from the group consisting of nitrogen, oxygen and/or sulfur in the ring; preferably is a heterocyclic ring system of one or two saturated or unsaturated rings of which at least one ring contains one or more heteroatoms selected from the group consisting of nitrogen, oxygen and/or sulfur in the ring, more preferably is selected from isothiazole, imidazole, oxadiazole, tetrazole, pyridine, pyrimidine, piperidine, piperazine, benzofuran, benzimidazole, indazole, benzothiazole, benzodiazole, thiazole, benzothiazole, tetrahydropyran, morpholine, indoline, furan, triazole, isoxazole, pyrazole, thiophene, benzothiophene, pyrrole, pyrazine, pyrrolo[2,3b]pyridine, quinoline, isoquinoline, phthalazine, benzo-1,2,5-thiadiazole, indole, benzotriazole, benzoxazole oxopyrrolidine, pyrimidine, benzodioxolane, benzodioxane, carbazole and quinazoline;

and/or

R₃ is selected from substituted or unsubstituted aryl and substituted or unsubstituted heterocyclyl;
wherein
the aryl is selected from phenyl, naphthyl, or anthracene; preferably is naphthyl and phenyl; more preferably is phenyl;
and/or
the heterocyclyl is a heterocyclic ring system of one or more saturated or unsaturated rings of which at least one ring contains one or more heteroatoms selected from the group consisting of nitrogen, oxygen and/or sulfur in the ring; preferably is a heterocyclic ring system of one or two saturated or unsaturated rings of which at least one ring contains one or more heteroatoms selected from the group consisting of nitrogen, oxygen and/or sulfur in the ring, more preferably is selected from isothiazole, imidazole, oxadiazole, tetrazole, pyridine, pyrimidine, piperidine, piperazine, benzofuran, benzimidazole, indazole, benzothiazole, benzodiazole, thiazole, benzothiazole, tetrahydropyran, morpholine, indoline, furan, triazole, isoxazole, pyrazole, thiophene, benzothiophene, pyrrole, pyrazine, pyrrolo[2,3b]pyridine, quinoline, isoquinoline, phthalazine, benzo-1,2,5-thiadiazole, indole, benzotriazole, benzoxazole oxopyrrolidine, pyrimidine, benzodioxolane, benzodioxane, carbazole and quinazoline;
and/or
R₄ is selected from hydrogen, unsubstituted C₁₋₆ alkyl, unsubstituted C₂₋₆ alkenyl and unsubstituted C₂₋₆ alkynyl;
wherein
the C₁₋₆ alkyl is preferably selected from methyl, ethyl, propyl, butyl, pentyl, hexyl, isopropyl, isobutyl and 2-methylpropyl, more preferably the C₁₋₆ alkyl is ethyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;

and/or

R_{4′} is selected from hydrogen, unsubstituted C₁₋₆ alkyl, unsubstituted C₂₋₆ alkenyl, unsubstituted C₂₋₆ alkynyl and -Boc;
wherein
the C₁₋₆ alkyl is preferably selected from methyl, ethyl, propyl, butyl, pentyl, hexyl, isopropyl, isobutyl and 2-methylpropyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;
and/or
R₅, R_{5′} and R_{5″} are independently selected from hydrogen, unsubstituted C₁₋₆ alkyl, unsubstituted C₂₋₆ alkenyl and unsubstituted C₂₋₆ alkynyl;
wherein
the C₁₋₆ alkyl is preferably selected from methyl, ethyl, propyl, butyl, pentyl, hexyl, isopropyl, isobutyl and 2-methylpropyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;
and/or
R_{5′″} is selected from hydrogen, unsubstituted C₁₋₆ alkyl, unsubstituted C₂₋₆ alkenyl, unsubstituted C₂₋₆ alkynyl and -Boc;
wherein
the C₁₋₆ alkyl is preferably selected from methyl, ethyl, propyl, butyl, pentyl, hexyl, isopropyl, isobutyl and 2-methylpropyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;
and/or
R₆, R_{6′} and R_{6″} are independently selected from hydrogen, unsubstituted C₁₋₆ alkyl, unsubstituted C₂₋₆ alkenyl and unsubstituted C₂₋₆ alkynyl;
wherein
the C₁₋₆alkyl is preferably selected from methyl, ethyl, propyl, butyl, pentyl, hexyl, isopropyl, isobutyl and 2-methylpropyl; more preferably the C₁₋₆alkyl is methyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;
and/or
R_{6′″} is selected from hydrogen, unsubstituted C₁₋₆ alkyl, unsubstituted C₂₋₆ alkenyl, unsubstituted C₂₋₆alkynyl and -Boc;
wherein
the C₁₋₆ alkyl is preferably selected from methyl, ethyl, propyl, butyl, pentyl, hexyl, isopropyl, isobutyl and 2-methylpropyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R₁, as defined in any of the embodiments of the present invention,

the C₁₋₆ alkyl is preferably selected from methyl, ethyl, propyl, butyl, pentyl, hexyl, isopropyl, isobutyl and 2-methylpropyl, more preferably the C₁₋₆ alkyl is methyl or ethyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R₂ as defined in any of the embodiments of the present invention,

the aryl is selected from phenyl, naphthyl, or anthracene; preferably is naphthyl and phenyl; more preferably is phenyl;
and/or
the heterocyclyl is a heterocyclic ring system of one or more saturated or unsaturated rings of which at least one ring contains one or more heteroatoms selected from the group consisting of nitrogen, oxygen and/or sulfur in the ring; preferably is a heterocyclic ring system of one or two saturated or unsaturated rings of which at least one ring contains one or more heteroatoms selected from the group consisting of nitrogen, oxygen and/or sulfur in the ring, more preferably is selected from isothiazole, imidazole, oxadiazole, tetrazole, pyridine, pyrimidine, piperidine, piperazine, benzofuran, benzimidazole, indazole, benzothiazole, benzodiazole, thiazole, benzothiazole, tetrahydropyran, morpholine, indoline, furan, triazole, isoxazole, pyrazole, thiophene, benzothiophene, pyrrole, pyrazine, pyrrolo[2,3b]pyridine, quinoline, isoquinoline, phthalazine, benzo-1,2,5-thiadiazole, indole, benzotriazole, benzoxazole oxopyrrolidine, pyrimidine, benzodioxolane, benzodioxane, carbazole and quinazoline;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R₃ as defined in any of the embodiments of the present invention,

the aryl is selected from phenyl, naphthyl, or anthracene; preferably is naphthyl and phenyl; more preferably is phenyl;
and/or
the heterocyclyl is a heterocyclic ring system of one or more saturated or unsaturated rings of which at least one ring contains one or more heteroatoms selected from the group consisting of nitrogen, oxygen and/or sulfur in the ring; preferably is a heterocyclic ring system of one or two saturated or unsaturated rings of which at least one ring contains one or more heteroatoms selected from the group consisting of nitrogen, oxygen and/or sulfur in the ring, more preferably is selected from isothiazole, imidazole, oxadiazole, tetrazole, pyridine, pyrimidine, piperidine, piperazine, benzofuran, benzimidazole, indazole, benzothiazole, benzodiazole, thiazole, benzothiazole, tetrahydropyran, morpholine, indoline, furan, triazole, isoxazole, pyrazole, thiophene, benzothiophene, pyrrole, pyrazine, pyrrolo[2,3b]pyridine, quinoline, isoquinoline, phthalazine, benzo-1,2,5-thiadiazole, indole, benzotriazole, benzoxazole oxopyrrolidine, pyrimidine, benzodioxolane, benzodioxane, carbazole and quinazoline;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R₄ as defined in any of the embodiments of the present invention,

the C₁₋₆ alkyl is preferably selected from methyl, ethyl, propyl, butyl, pentyl, hexyl, isopropyl, isobutyl and 2-methylpropyl, more preferably the C₁₋₆ alkyl is ethyl;

and/or
the C₂₋₆-alkenyl is preferably selected from ethylene, propylene, butylene, pentylene, hexylene, isopropylene and isobutylene;

and/or

the C₂₋₆-alkynyl is preferably selected from ethyne, propyne, butyne, pentyne, hexyne, isopropyne and isobutyne;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R_{4′} as defined in any of the embodiments of the present invention,

the C₁₋₆ alkyl is preferably selected from methyl, ethyl, propyl, butyl, pentyl, hexyl, isopropyl, isobutyl and 2-methylpropyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R₅, R_{5′} and R_{5″} as defined in any of the embodiments of the present invention,

the C₁₋₆ alkyl is preferably selected from methyl, ethyl, propyl, butyl, pentyl, hexyl, isopropyl, isobutyl and 2-methylpropyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R_{5′″} as defined in any of the embodiments of the present invention,

the C₁₋₆ alkyl is preferably selected from methyl, ethyl, propyl, butyl, pentyl, hexyl, isopropyl, isobutyl and 2-methylpropyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R₆, R_{6′} and R_{6″} as defined in any of the embodiments of the present invention,

the C₁₋₆ alkyl is preferably selected from methyl, ethyl, propyl, butyl, pentyl, hexyl, isopropyl, isobutyl and 2-methylpropyl; more preferably the C₁₋₆ alkyl is methyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R_{6′″} as defined in any of the embodiments of the present invention,

the C₁₋₆ alkyl is preferably selected from methyl, ethyl, propyl, butyl, pentyl, hexyl, isopropyl, isobutyl and 2-methylpropyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein

m is 0, 1, 2, 3, 4 or 5; preferably m is 0 or 1;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein

n is 1, 2, 3, 4 or 5; preferably n is 1 or 2;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein

X is a group selected from pyrimidine, pyrazine, oxadiazole, thiazole, thiadiazole, triazole and indazole;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I), the compound is a compound of Formula (I^{5′})

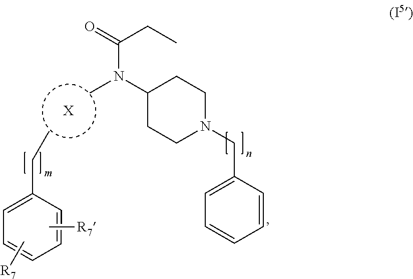

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I), the compound is a compound of Formula (I^{5′})

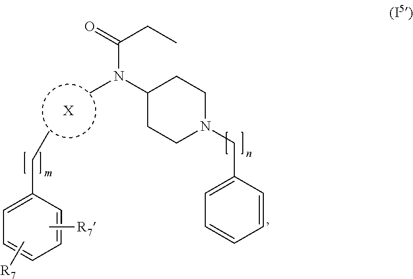

wherein
m is 0, 1, 2, 3, 4 or 5;
n is 1, 2, 3, 4 or 5;
X is a group selected from pyrimidine, pyrazine, oxadiazole, thiazole, thiadiazole, triazole and indazole;
R₇ and R_{7′} are independently selected from halogen, —R₆, —OR₆, —NO₂, —NR₆R_{6′″}, NR₆C(O)R_{6′}, —NR₆S(O)₂R_{6′}, —S(O)₂NR₆R_{6′}, —NR₆C(O)NR_{6′}R_{6″}, —SR₆, —S(O)R₆, S(O)₂R₆, —CN, haloalkyl, haloalkoxy, —C(O)OR₆, —C(O)NR₆R_{6′}, —OCH₂CH₂OH, —NR₆S(O)₂NR_{6′}R_{6″} and —C(CH₃)₂OR₆;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further preferred embodiment of the invention according to general Formula (I) the compound is a compound of Formula (I^{6′}),

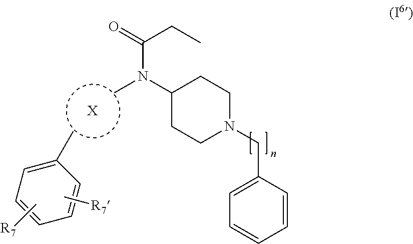

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further preferred embodiment of the invention according to general Formula (I) the compound is a compound of Formula (I^{6′}),

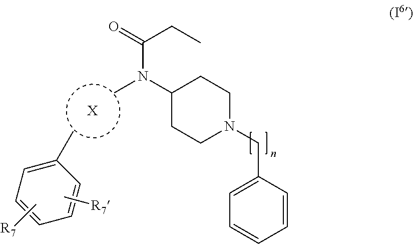

n is 1, 2, 3, 4 or 5;
X is a group selected from pyrimidine, pyrazine, oxadiazole, thiazole, thiadiazole, triazole and indazole;
R₇ and R_{7′} are independently selected from halogen, —R₆, —OR₆, —NO₂, —NR₆R_{6′″}, NR₆C(O)R_{6′}, —NR₆S(O)₂R_{6′}, —S(O)₂NR₆R_{6′}, —NR₆C(O)NR_{6′}R_{6″}, —SR₆, —S(O)R₆, S(O)₂R₆, —CN, haloalkyl, haloalkoxy, —C(O)OR₆, —C(O)NR₆R_{6′}, —OCH₂CH₂OH, —NR₆S(O)₂NR_{6′}R_{6″} and —C(CH₃)₂OR₆;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a preferred embodiment

X is a group selected from pyrimidine, pyrazine, oxadiazole, thiazole, thiadiazole, triazole and indazole;

In a preferred embodiment

X is a group selected from

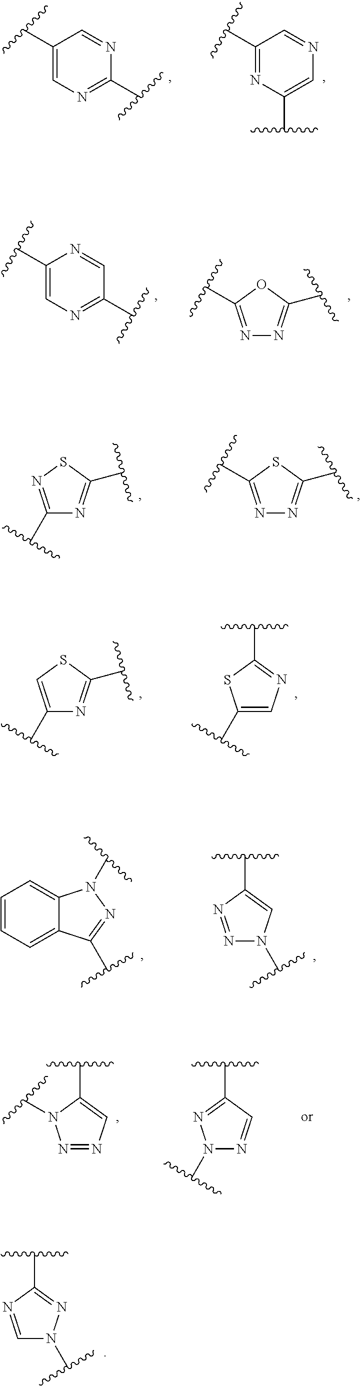

In a preferred embodiment

the pyrimidine in X is

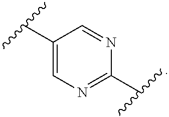 [SMILES: *c1cnc(*)nc1]

In a preferred embodiment

the pyrimidine in X is

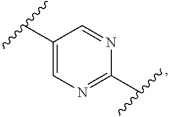 [SMILES: *c1cnc(*)nc1]

leading to compound of formula (Ia1)

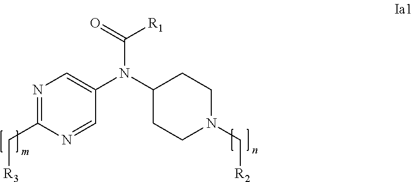

In a preferred embodiment

the pyrazine in X is

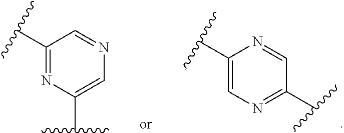 [SMILES: *c1cncc(*)n1 ; *c1cnc(*)cn1]

In a preferred embodiment

the pyrazine in X is

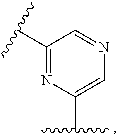 [SMILES: *c1cncc(*)n1]

leading to compound of formula (Ia2)

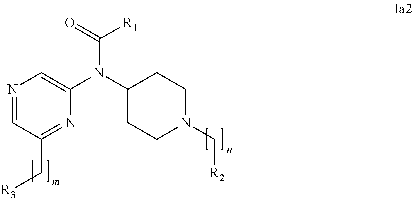

In a preferred embodiment

the pyrazine in X is o

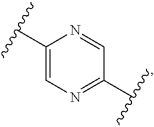 [SMILES: *c1cnc(*)cn1]

leading to compound of formula (Ia3)

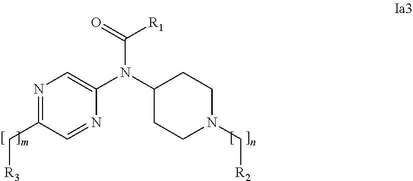

In a preferred embodiment

the oxadiazole in X is

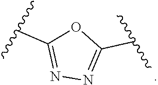 [SMILES: *c1nnc(*)o1]

In a preferred embodiment

the oxadiazole in X is

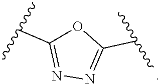 [SMILES: *c1nnc(*)o1]

leading to a compound of formula (Ia4)

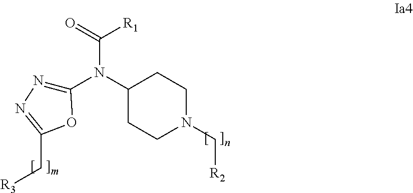

In a preferred embodiment

the thiazole in X is

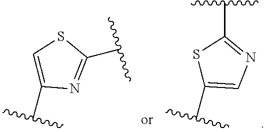 [SMILES: *c1csc(*)n1 ; *c1cnc(*)s1]

In a preferred embodiment

the thiazole in X is

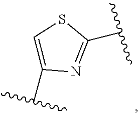 [SMILES: *c1csc(*)n1]

leading to a compound of formula (Ia5)

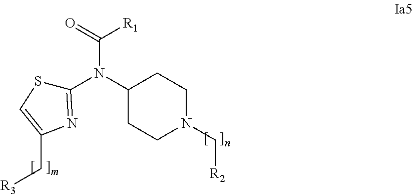

In a preferred embodiment

the thiazole in X is

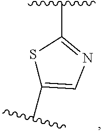 [SMILES: *c1cnc(*)s1]

leading to a compound of formula (Ia6)

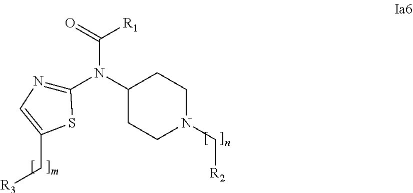

In a preferred embodiment

the thiadiazole in X is

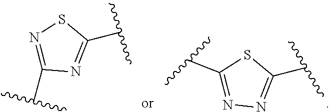 [SMILES: *c1nsc(*)n1 ; *c1nnc(*)s1]

In a preferred embodiment

the thiadiazole in X is

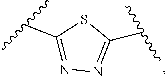 [SMILES: *c1nnc(*)s1]

leading to a compound of formula (Ia7)

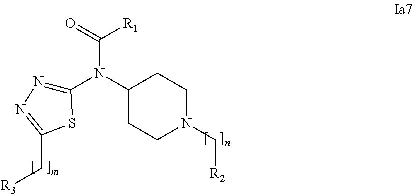

In a preferred embodiment

the thiadiazole in X is

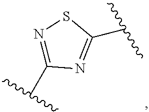 [SMILES: *c1nsc(*)n1]

leading to a compound of formula (Ia8)

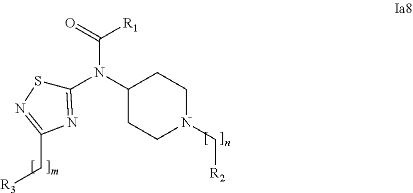

In a preferred embodiment the triazole in X is

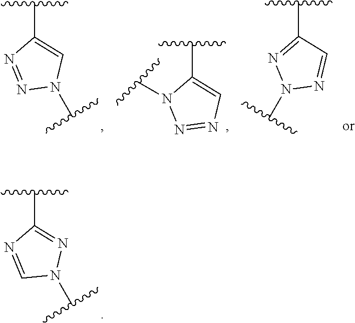 [SMILES: *c1cn(*)nn1 ; *c1cnnn1* ; *c1cnn(*)n1 ; *c1ncn(*)n1]

In a preferred embodiment

the triazole in X is

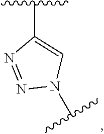 [SMILES: *c1cn(*)nn1]

leading to a compound of formula (Ia9)

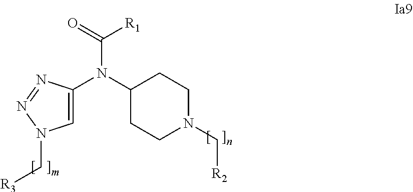

In a preferred embodiment

the triazole in X is

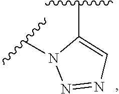 [SMILES: *c1cnnn1*]

leading to a compound of formula (Ia10)

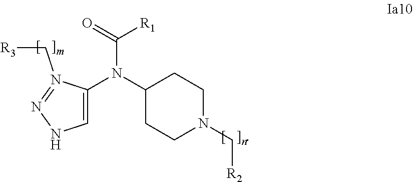

In a preferred embodiment

the triazole in X is

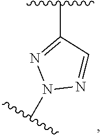 [SMILES: *c1cnn(*)n1]

leading to a compound of formula (Ia11)

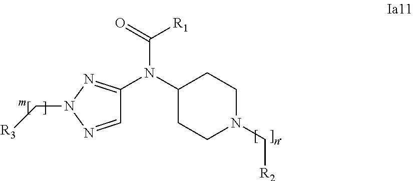

In a preferred embodiment

the triazole in X is

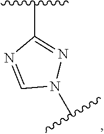 [SMILES: *c1ncn(*)n1]

leading to a compound of formula (Ia12)

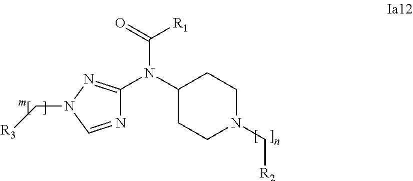

In a preferred embodiment

the indazole in X is

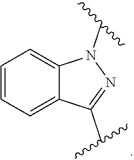 [SMILES: *c1nn(*)c2ccccc12]

In a preferred embodiment

the indazole in X is

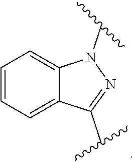 [SMILES: *c1nn(*)c2ccccc12]

leading to a compound of formula (Ia13)

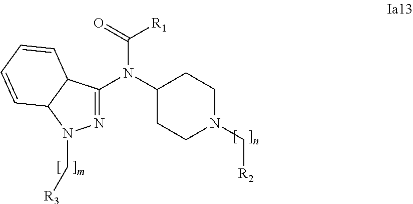

In a preferred embodiment

R₁ is a substituted or unsubstituted group selected from methyl, ethyl and —CH(CH₃)C(O)-ethyl, preferably is a unsubstituted group selected from methyl, ethyl and —CH(CH₃)C(O)-ethyl.

In a preferred embodiment

R₂ is substituted or unsubstituted phenyl, preferably R₂ is unsubstituted phenyl.

In a preferred embodiment

R₃ is substituted or unsubstituted phenyl.

In a preferred embodiment

R₄ is substituted or unsubstituted ethyl, preferably unsubstituted ethyl.

In a preferred embodiment

R₆ is hydrogen or substituted or unsubstituted methyl, preferably is hydrogen or unsubstituted methyl.

In a preferred embodiment

R₇ is fluorine, chlorine, —OH or substituted or unsubstituted —O-methyl, preferably is fluorine, chlorine, —OH or unsubstituted —O-methyl.

In another preferred embodiment

n is 1 or 2;

In another preferred embodiment

m is 0 or 1;

In an particular embodiment

the halogen is fluorine or chlorine, bromine or iodine, preferably is fluorine or chlorine.

In a preferred further embodiment, the compounds of the general Formula (I) are selected from

| EX | Chemical name |
|---|---|
| 1 | N-(1-benzylpiperidin-4-yl)-N-(2-(4-fluorophenyl)pyrimidin-5-yl)propionamide |
| 2 | N-(1-benzylpiperidin-4-yl)-N-(6-(4-fluorophenyl)pyrazin-2-yl)propionamide |
| 3 | N-(1-benzylpiperidin-4-yl)-N-(5-(4-fluorophenyl)pyrazin-2-yl)propionamide |
| 4 | N-(1-benzylpiperidin-4-yl)-N-(5-phenyl-1,3,4-oxadiazol-2-yl)propionamide |
| 5 | N-(1-benzylpiperidin-4-yl)-N-(3-phenyl-1,2,4-thiadiazol-5-yl)propionamide |
| 6 | N-(1-benzylpiperidin-4-yl)-N-(5-phenyl-1,3,4-thiadiazol-2-yl)propionamide |
| 7 | N-(1-benzylpiperidin-4-yl)-N-(4-phenylthiazol-2-yl)propionamide |
| 8 | N-(1-benzylpiperidin-4-yl)-2-methyl-3-oxo-N-(4-phenylthiazol-2-yl)pentanamide |
| 9 | N-(1-benzylpiperidin-4-yl)-N-(5-phenylthiazol-2-yl)propionamide |
| 10 | N-(2-(4-fluorophenyl)pyrimidin-5-yl)-N-(1-phenethylpiperidin-4-yl)propionamide |
| 11 | N-(6-(4-fluorophenyl)pyrazin-2-yl)-N-(1-phenethylpiperidin-4-yl)propionamide |
| 12 | N-(5-(4-fluorophenyl)pyrazin-2-yl)-N-(1-phenethylpiperidin-4-yl)propionamide |
| 13 | N-(1-benzylpiperidin-4-yl)-N-(1-phenyl-1H-indazol-3-yl)propionamide |
| 14 | N-(1-benzylpiperidin-4-yl)-N-(1-(4-chlorophenyl)-1H-indazol-3-yl)propionamide |
| 15 | N-(1-benzylpiperidin-4-yl)-N-(1-(4-fluorophenyl)-1H-indazol-3-yl)propionamide |
| 16 | N-(1-benzylpiperidin-4-yl)-N-(1-(3-chloro-4-fluorophenyl)-1H-indazol-3-yl)propionamide |
| 17 | N-(1-benzylpiperidin-4-yl)-N-(1-(4-methoxyphenyl)-1H-indazol-3-yl)propionamide |
| 18 | N-(1-benzylpiperidin-4-yl)-N-(1-(4-hydroxyphenyl)-1H-indazol-3-yl)propionamide |
| 19 | N-(1-benzylpiperidin-4-yl)-N-(1-(4-fluorophenyl)-1H-1,2,3-triazol-4-yl)propionamide |
| 20 | N-(1-(4-fluorophenyl)-1H-1,2,3-triazol-4-yl)-N-(1-phenethylpiperidin-4-yl)propionamide |
| 21 | N-(1-(3,4-dichlorobenzyl)-1H-1,2,3-triazol-4-yl)-N-(1-phenethylpiperidin-4-yl)propionamide |
| 22 | N-(1-(4-chloro-3-fluorophenyl)-1H-1,2,3-triazol-4-yl)-N-(1-phenethylpiperidin-4-yl)propionamide |
| 23 | N-(1-(3,4-dichlorobenzyl)-1H-1,2,3-triazol-4-yl)-N-(1-phenethylpiperidin-4-yl)acetamide |
| 24 | N-(1-benzylpiperidin-4-yl)-N-(1-(4-methoxybenzyl)-1H-1,2,3-triazol-4-yl)propionamide |
| 25 | N-(1-(4-methoxybenzyl)-1H-1,2,3-triazol-4-yl)-N-(1-phenethylpiperidin-4-yl)propionamide |
| 26 | N-(1-(4-fluorophenyl)-1H-1,2,3-triazol-5-yl)-N-(1-phenethylpiperidin-4-yl)propionamide |
| 27 | N-(2-(4-fluorophenyl)-2H-1,2,3-triazol-4-yl)-N-(1-phenethylpiperidin-4-yl)propionamide |
| 28 | N-(1-benzylpiperidin-4-yl)-N-(2-(4-fluorophenyl)-2H-1,2,3-triazol-4-yl)propionamide |
| 29 | N-(1-benzylpiperidin-4-yl)-N-(1-phenyl-1H-1,2,4-triazol-3-yl)propionamide |
| 30 | N-(1-benzylpiperidin-4-yl)-N-(1-(4-fluorophenyl)-1H-1,2,4-triazol-3-yl)propionamide |

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a preferred embodiment of the compound according to the invention of general Formula (I),

R₁ is selected from substituted or unsubstituted C₁₋₆ alkyl, substituted or unsubstituted C₂₋₆ alkenyl, and substituted or unsubstituted C₂₋₈ alkynyl;
wherein the alkyl, alkenyl or alkynyl in R₁, if substituted, is substituted with one or more substituent/s selected from —OR₄, —C(O)R₄, halogen, —CN, C₁₋₄ haloalkyl, C₁₋₄ haloalkoxy and —NR₄R_{4′};
wherein R₄ is selected from hydrogen, unsubstituted C₁₋₆ alkyl, unsubstituted C₂₋₆ alkenyl and unsubstituted C₂₋₆ alkynyl;
and R_{4′} is selected from hydrogen, unsubstituted C₁₋₆ alkyl, unsubstituted C₂₋₆ alkenyl, unsubstituted C₂₋₆ alkynyl and -Boc;
   optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a preferred embodiment of the compound according to the invention of general Formula (I),

R₂ is selected from substituted or unsubstituted aryl and substituted or unsubstituted heterocyclyl;
wherein said aryl or heterocyclyl in R₂, if substituted, is substituted with one or more substituent/s selected from halogen, —R₅, —OR₅, —NO₂, —NR₅R_{5′″}, NR₅C(O)R_{5′}, —NR₅(O)₂R_{5′}, —S(O)₂NR₅R_{5′}, —NR₅C(O)NR_{5′}R_{5″}, —SR₅, —S(O)R₅, S(O)₂R₅, —CN, C₁₋₄ haloalkyl, C₁₋₄ haloalkoxy, —C(O)OR₅, —C(O)NR₅R_{5′}, —OCH₂CH₂OH, —NR₅S(O)₂NR_{5′}R_{5″} and C(CH₃)₂OR₅;
and wherein the non-aromatic heterocyclyl in R₂, if substituted, may also be spirosubstituted with

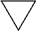

or substituted with ═O;
wherein R₅, R_{5′} and R_{5″} are independently selected from hydrogen, unsubstituted C₁₋₆ alkyl, unsubstituted C₂₋₆ alkenyl and unsubstituted C₂₋₆ alkynyl;
and wherein R_{5′″} is selected from hydrogen, unsubstituted C₁₋₆ alkyl, unsubstituted C₂₋₆ alkenyl, unsubstituted C₂₋₆ alkynyl and -Boc;
   optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.
   R₂ is selected from substituted or unsubstituted aryl and substituted or unsubstituted heterocyclyl;
wherein said aryl or heterocycyl in R₂, if substituted, is substituted with one or more substituent/s selected from halogen, —R₅, —OR₅, —NO₂, —NR₅R_{5′″}, NR₅C(O)R_{5′}, —NR₅(O)₂R_{5′}, —S(O)₂NR₅R_{5′}, —NR₅C(O)NR_{5′}R_{5″}, —SR₅, —S(O)R₅, S(O)₂R₅, —CN, C₁₋₄ haloalkyl, C₁₋₄ haloalkoxy, —C(O)OR₅, —C(O)NR₅R_{5′}, —OCH₂CH₂OH, —NR₅S(O)₂NR_{5′}R_{5″} and C(CH₃)₂OR₅;
and wherein the non-aromatic heterocycyl in R₂, if substituted, may also be spirosubstituted or substituted with ═O;
wherein R₅, R_{5′}, and R_{5″} are independently selected from hydrogen, unsubstituted C₁₋₆ alkyl, unsubstituted C₂₋₆ alkenyl and unsubstituted C₂₋₆ alkynyl;
and wherein R_{5′″} is selected from hydrogen, unsubstituted C₁₋₆ alkyl, unsubstituted C₂₋₆ alkenyl, unsubstituted C₂₋₆ alkynyl and -Boc;
   optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another embodiment of the invention the compound of general Formula (I),

R₃ is selected from substituted or unsubstituted aryl and substituted or unsubstituted heterocyclyl;
wherein said aryl or heterocyclyl in R₃, if substituted, is substituted with one or more substituent/s selected from halogen, —R₆, —OR₆, —NO₂, —NR₆R_{6′″}, NR₆C(O)R_{6′}, —NR₆(O)₂R_{6′}, —S(O)₂NR₆R_{6′}, —NR₆C(O)NR_{6′}R_{6″}, —SR₆, —S(O)R₆, S(O)₂R₆, —CN, C₁₋₄ haloalkyl, C₁₋₄ haloalkoxy, —C(O)OR₆, —C(O)NR₆R_{6′}, —OCH₂CH₂OH, —NR₆S(O)₂NR_{6′}R_{6″} and C(CH₃)₂OR₆;
and wherein a non-aromatic heterocyclyl in R₃, if substituted, may also be spirosubstituted with

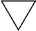

or substituted with ═O;
wherein R₆, R_{6′}, and R_{6″} are independently selected from hydrogen, unsubstituted C₁₋₆ alkyl, unsubstituted C₂₋₆ alkenyl and unsubstituted C₂₋₆ alkynyl;
and wherein R_{6′″} is selected from hydrogen, unsubstituted C₁₋₆ alkyl, unsubstituted C₂₋₆ alkenyl, unsubstituted C₂₋₆ alkynyl and -Boc;
   optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another embodiment of the invention the compound of general Formula (I),

R₃ is selected from substituted or unsubstituted aryl and substituted or unsubstituted heterocyclyl;
wherein said aryl or heterocyclyl in R₃, if substituted, is substituted with one or more substituent/s selected from halogen, —R₆, —OR₆, —NO₂, —NR₆R_{6′″}, NR₆C(O)R_{6′}, —NR₆(O)₂R_{6′}, —S(O)₂NR₆R_{6′}, —NR₆C(O)NR_{6′}R_{6″}, —SR₆, —S(O)R₆, S(O)₂R₆, —CN, C₁₋₄ haloalkyl, C₁₋₄ haloalkoxy, —C(O)OR₆, —C(O)NR₆R_{6′}, —OCH₂CH₂OH, —NR₆S(O)₂NR_{6′}R_{6″} and C(CH₃)₂OR₆;
and wherein a non-aromatic heterocyclyl in R₃, if substituted, may also be spirosubstituted or substituted with ═O;
wherein R₆, R_{6′} and R_{6″} are independently selected from hydrogen, unsubstituted C₂₋₆ alkyl, unsubstituted C₂₋₆ alkenyl and unsubstituted C₂₋₆ alkynyl;
and wherein R_{6′″} is selected from hydrogen, unsubstituted C₁₋₆ alkyl, unsubstituted C₂₋₆ alkenyl, unsubstituted C₂₋₆ alkynyl and -Boc;
   optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a preferred embodiment of the compound according to the invention of general Formula (I) and in relation to R₁ of any of the embodiments of the present invention,

the alkyl, alkenyl or alkynyl in R₁, if substituted, is substituted with one or more substituent/s selected from —OR₄, —C(O)R₄, halogen, —CN, C₁₋₄ haloalkyl, C₁₋₄ haloalkoxy and —NR₄R_{4′};
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a preferred embodiment of the compound according to the invention of general Formula (I) and in relation to R₁ of any of the embodiments of the present invention,

the alkyl, alkenyl or alkynyl in R₁, if substituted, is substituted with —C(O)R₄;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a preferred embodiment of the compound according to the invention of general Formula (I) and in relation to R₁ of any of the embodiments of the present invention,

the alkyl, alkenyl or alkynyl in R₁, if substituted, is substituted with one or more substituent/s selected from —C(O)-ethyl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a preferred embodiment of the compound according to the invention of general Formula (I) and in relation to R₂ of any of the embodiments of the present invention,
Wherein said aryl or heterocyclyl in R₂, if substituted, is substituted with one or more substituent/s selected from halogen, —R₅, —OR₅, —NO₂, —NR₅R_{5′″}, NR₅C(O)R_{5′}, —NR₅(O)₂R_{5′}, —S(O)₂NR₅R_{5′}, —NR₅C(O)NR_{5′}R_{5″}, —SR₅, —S(O)R₅, S(O)₂R₅, —CN, C₁₋₄ haloalkyl, C₁₋₄ haloalkoxy, —C(O)OR₅, —C(O)NR₅R_{5′}, —OCH₂CH₂OH, —NR₅S(O)₂NR_{5′}R_{5″} and C(CH₃)₂OR₅;
and wherein the non-aromatic heterocyclyl in R₂, if substituted, may also be spirosubstituted with

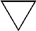

or substituted with ═O;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a preferred embodiment of the compound according to the invention of general Formula (I) and in relation to R₂ of any of the embodiments of the present invention,
Wherein said aryl or heterocyclyl in R₂, if substituted, is substituted with one or more substituent/s selected from halogen, —R₅, —OR₅, —NO₂, —NR₅R_{5′″}, NR₅C(O)R_{5′}, —NR₅(O)₂R_{5′}, —S(O)₂NR₅R_{5′}, —NR₅C(O)NR_{5′}R_{5″}, —SR₅, —S(O)R₅, S(O)₂R₅, —CN, C₁₋₄ haloalkyl, C₁₋₄ haloalkoxy, —C(O)OR₅, —C(O)NR₅R_{5′}, —OCH₂CH₂OH, —NR₅S(O)₂NR_{5′}R_{5″} and C(CH₃)₂OR₅;
and wherein the non-aromatic heterocyclyl in R₂, if substituted, may also be spirosubstituted or substituted with ═O;
   optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a preferred embodiment of the compound according to the invention of general Formula (I) and in relation to R₃ of any of the embodiments of the present invention,
Aryl or heterocyclyl in R₃, if substituted, is substituted with one or more substituent/s selected from halogen, —R₆, —OR₆, —NO₂, —NR₆R_{6′″}, —NR₆C(O)R_{6′}, —NR₆(O)₂R_{6′}, —S(O)₂NR₆R_{6′}, —NR₆C(O)NR_{6′}R_{6″}, —SR₆, —S(O)R₆, S(O)₂R₆, —CN, C₁₋₄ haloalkyl, C₁₋₄ haloalkoxy, —C(O)OR₆, —C(O)NR₆R_{6′}, —OCH₂CH₂OH, —NR₆S(O)₂NR_{6′}R_{6″} and C(CH₃)₂OR₆;
and wherein a non-aromatic heterocyclyl in R₃, if substituted, may also be spirosubstituted with

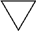

or substituted with ═O;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a preferred embodiment of the compound according to the invention of general Formula (I) and in relation to R₃ of any of the embodiments of the present invention,
Aryl or heterocyclyl in R₃, if substituted, is substituted with one or more substituent/s selected from halogen, —R₆, —OR₆, —NO₂, —NR₆R_{6′″}, —NR₆C(O)R_{6′}, —NR₆(O)₂R_{6′}, —S(O)₂NR₆R_{6′}, —NR₆C(O)NR_{6′}R_{6″}, —SR₆, —S(O)R₆, S(O)₂R₆, —CN, C₁₋₄ haloalkyl, C₁₋₄ haloalkoxy, —C(O)OR₆, —C(O)NR₆R_{6′}, —OCH₂CH₂OH, —NR₆S(O)₂NR_{6′}R_{6″} and C(CH₃)₂OR₆;
and wherein a non-aromatic heterocyclyl in R₃, if substituted, may also be spirosubstituted or substituted with ═O;
   optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a preferred embodiment of the compound according to the invention of general Formula (I) and in relation to R₃ of any of the embodiments of the present invention,

aryl or heterocyclyl in R₃, if substituted, is substituted with one or more substituent/s selected from halogen and —OR₆;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a preferred embodiment of the compound according to the invention of general Formula (I) and in relation to R₃ of any of the embodiments of the present invention,

aryl or heterocyclyl in Rₐ, if substituted, is substituted with one or more substituent/s selected from fluorine, chloride, —OH, methoxy.
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In an embodiment of the compound according to the invention of general Formula (I),

the halogen is fluorine, chlorine, iodine or bromine;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a most preferred embodiment of the compound according to the invention of general Formula (I)

the halogen is fluorine or chlorine;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In an embodiment of the compound according to the invention of general Formula (I),

the haloalkyl is —CF₃;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another embodiment of the compound according to the invention of general Formula (I),

the haloalkoxy is —OCF₃;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

As this invention is aimed at providing a compound or a chemically related series of compounds which act as dual ligands of the σ₁ receptor and the μ-opioid receptor it is a very preferred embodiment in which the compounds are selected which act as dual ligands of the σ₁ receptor and the μ-opioid receptor and especially compounds which have a binding expressed as Kᵢ which is preferably <1000 nM for both receptors, more preferably <500 nM, even more preferably <100 nM.

In the following the phrase “compound of the invention” is used. This is to be understood as any compound according to the invention as described above according to general Formula (I), (I⁵′) or (I⁶′) or to to general Formula (I′), (I²′), (I³′) or (I⁴′).

The compounds of the invention represented by the above described Formula (I) may include enantiomers depending on the presence of chiral centres or isomers depending on the presence of multiple bonds (e.g. Z, E). The single isomers, enantiomers or diastereoisomers and mixtures thereof fall within the scope of the present invention.

For the sake of clarity the expression “a compound according to Formula (I), wherein R₁, R₂, R₃, R₄, etc. are as defined below in the detailed description” would (just like the expression e.g. “a compound of Formula (I) as defined in any one of claims 1 to 10” found in the claims) refer to “a compound according to Formula (I)”, wherein the definitions of the respective substituents R₁ etc. (also from the cited claims) are applied. In addition, this would also mean, though (especially in regards to the claims) that also one or more disclaimers defined in the description (or used in any of the cited claims like e.g. claim 1) would be applicable to define the respective compound. Thus, a disclaimer found in e.g. claim 1 would be also used to define the compound “of Formula (I) as defined in any one of claims 1 to 10”.

In general the processes are described below in the experimental part. The starting materials are commercially available or can be prepared by conventional methods.

A preferred aspect of the invention is also a process for the production of a compound according to Formula (I), following scheme 1.

A preferred embodiment of the invention is a process for the production of a compound according to Formula (I), wherein R₁, R₂, R₃, X, m and n are as defined in the description, following scheme 1.

In all processes and uses described underneath and in scheme 1, the values of R₁, R₂, R₃, X, m and n are as defined in the description, wherein L is a leaving group such as halogen, mesylate, tosylate or triflate and Z is chloro, bromo, methoxy or ethoxy, Y is

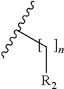

(the group indicated in a square in Scheme 1) and PG is a protecting group.

In a particular embodiment there is a process for the production of a compound of Formula (I),

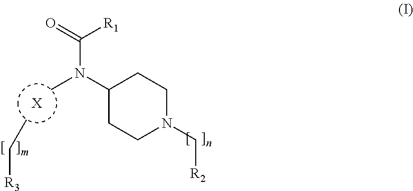

said process comprises the acylation of compounds of formula IVb

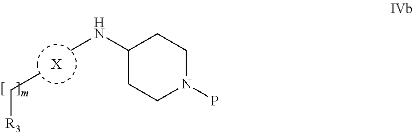

where P represents the following moiety

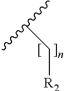

with a compound of formula VIa or a compound formula VIb

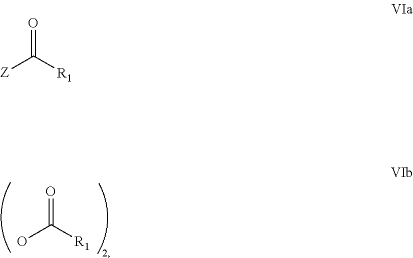

where Z represents a suitable leaving group, preferably an halogen or an ethoxy or methoxy group,

This reaction is represented in step 2 of scheme 1 below.

In a particular embodiment there is a process for the production of a compound of Formula (I),

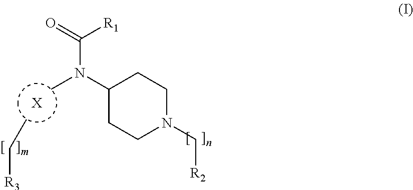

said process comprises the alkylation of a compound of Formula VIII,

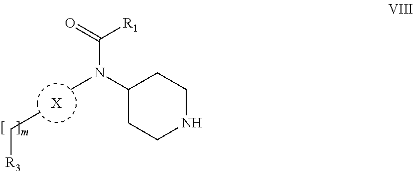

with a compound of formula IXa,

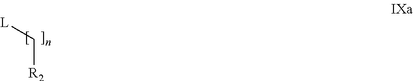

where L is a suitable leaving group, preferably a halogen, mesylate, tosylate or triflate

This reaction is represented in step 4 of scheme 1.

In a particular embodiment there is a process for the production of a compound of Formula (I),

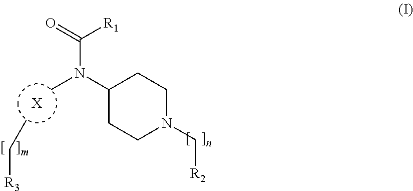

said process comprises the reductive amination reaction between a compound of formula VIII,

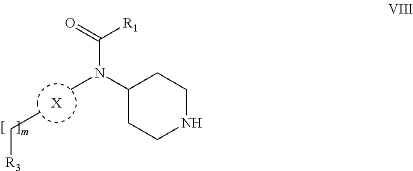

and a compound of formula IXb,

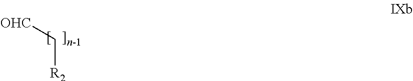

This reaction is represented in step 4 of scheme 1.

In a particular embodiment there is a process for the production of a compound of Formula (I), in the case wherein

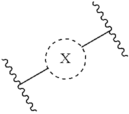

is triazole

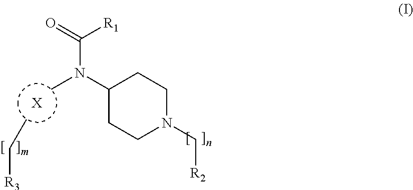

said process comprises the reaction of the compound XIb

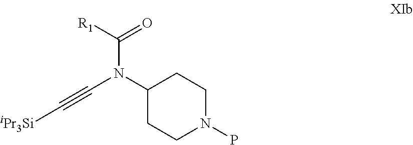

where P represents the moiety Y

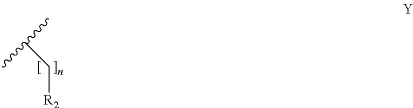

with azide derivatives of formula XII

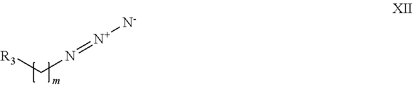

This reaction is represented in scheme 1

In a particular embodiment there is a process for the production of a compound of Formula (I),

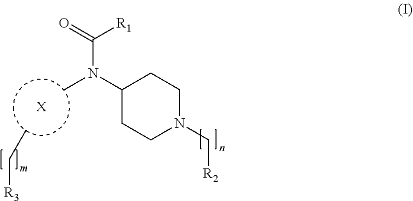

said process comprises the acylation of compounds of formula IVb

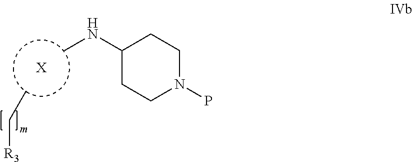

where P represents the following moiety

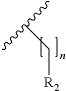

with a compound of formula VIa or a compound formula VIb

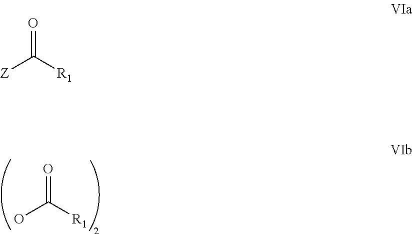

where Z represents a suitable leaving group, preferably an halogen or an ethoxy or methoxy group,
or
said process comprises the alkylation of a compound of Formula VIII,

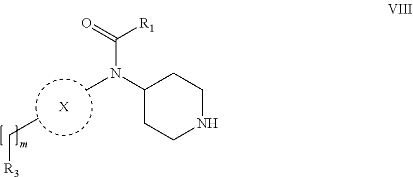

with a compound of formula IXa,

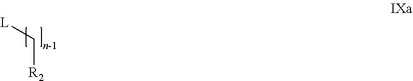

where L is a suitable leaving group, preferably a halogen, mesylate, tosylate or triflate
or
said process comprises the reductive amination reaction between a compound of formula VIII,

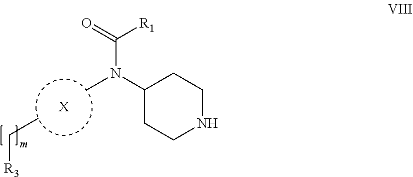

and a compound of formula IXb,

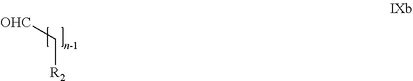

or
in the case where in formula I

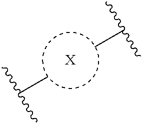

is triazole
said process comprises the reaction of the compound XIb

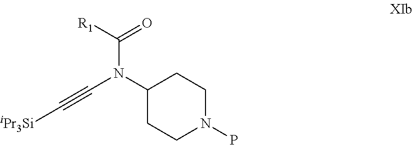

where P represents the moiety Y

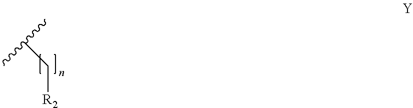

with azide derivatives of formula XII

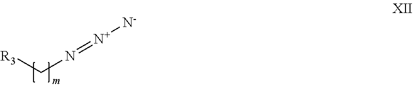

where X, R₁, R₂, R₃, n and m are as defined in the description.

The chemical reactions of the different compounds and intermediates for the preparation of compounds of formula (I) are summarized in the following paragraphs.

In a particular embodiment there is a process for the production of a compound (I) or (VII) starting from a compound (IVb) or (IVa), respectively,

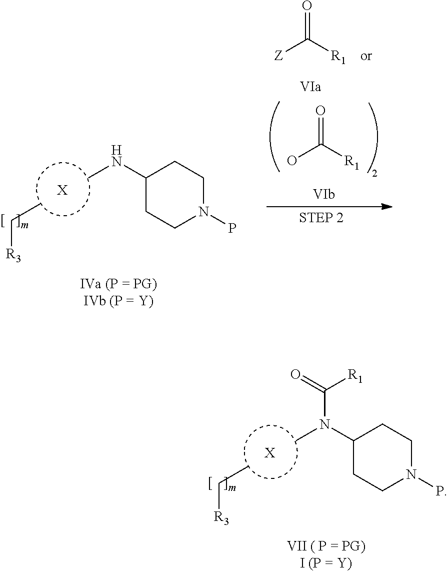

In a particular embodiment there is a process for the production of a compound (IVa) or (IVb) starting from a compound (IIa),

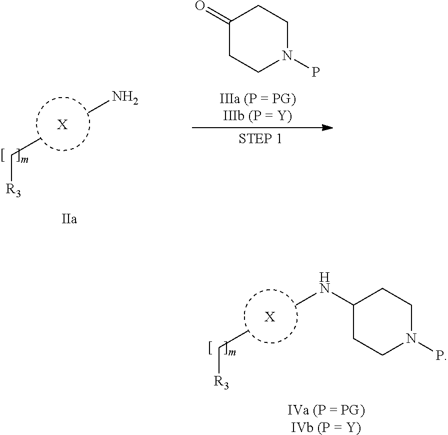

In a particular embodiment there is a process for the production of a compound (IVa) or (IVb) starting with a compound (IIb),

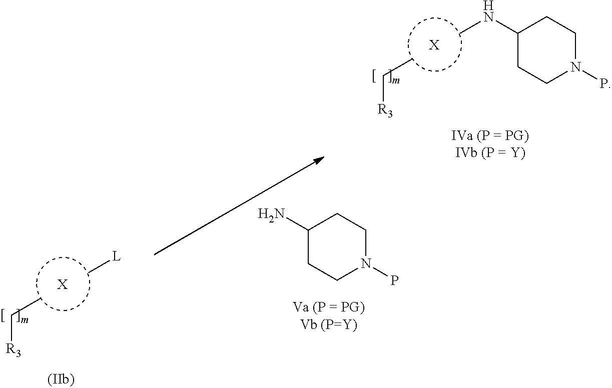

In a particular embodiment there is a process for the production of a compound (Xa) or (Xb) starting with a compound (Va) or (Vb), respectively,

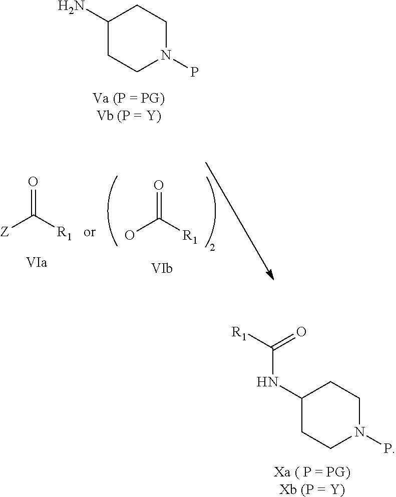

In a particular embodiment there is a process for the production of a compound (XIa) or (XIb) starting with a compound (Xa) or (Xb), respectively,

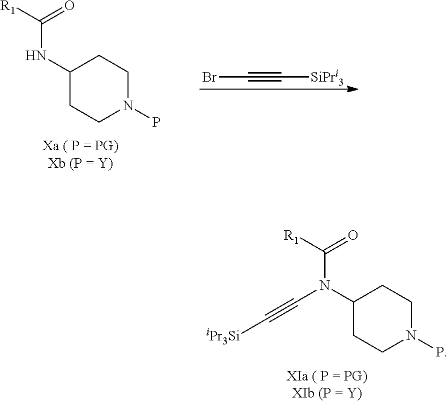

In a particular embodiment there is a process for the production of a compound (VII) or (I) starting with a compound (XIa) or (XIb), respectively,

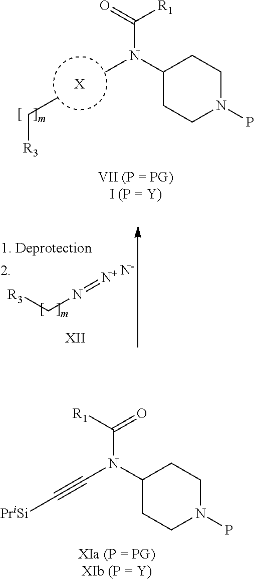

In a particular embodiment there is a process for the production of a compound (I) by deprotection of a compound (VII) followed by either an alkylation of a compound (IXa) or by reductive amination reaction between a compound (VIII) and a compound (IXb),

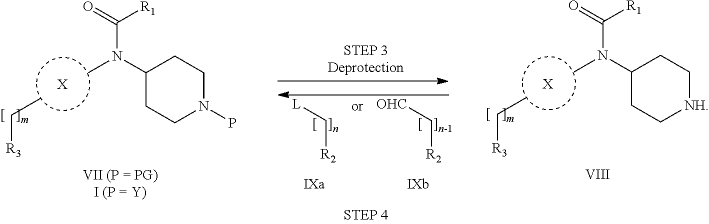

The different intermediates are used in the context of the invention to prepare compounds of formula (I)

In particular embodiment a compound of Formula (IIa),

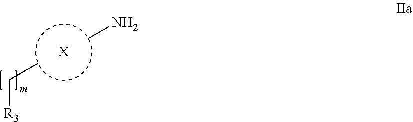

is used for the preparation of a compound of Formula (I).

In another particular embodiment a compound of Formula (IIb),

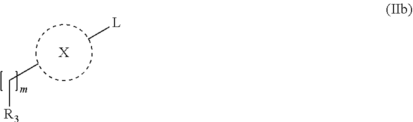

is used for the preparation of a compound of Formula (I).

In another particular embodiment a compound of Formula (IIIa) or (IIIb),

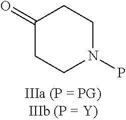

is used for the preparation of a compound of Formula (I).

In another particular embodiment a compound of Formula (IVa) or (IVb),

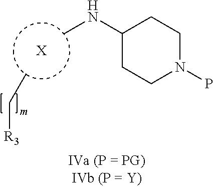

is used for the preparation of a compound of Formula (I).

In another particular embodiment a compound of Formula (Va) or (Vb),

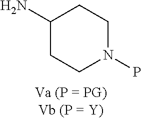

is used for the preparation of a compound of Formula (I).

In another particular embodiment a compound of Formula (VIa) or (VIb),

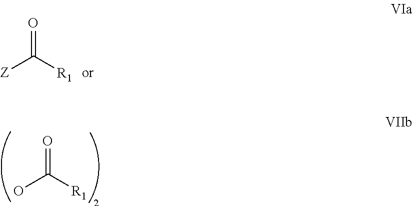

is used for the preparation of a compound of Formula (I).

In another particular embodiment a compound of Formula (VII),

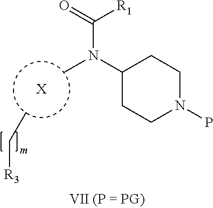

is used for the preparation of a compound of Formula (I).

In another particular embodiment a compound of Formula (VIII),

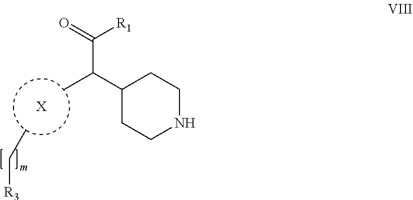

is used for the preparation of a compound of Formula (I).

In another particular embodiment a compound of Formula (IXa),

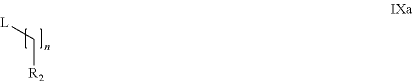

is used for the preparation of a compound of Formula (I).

In another particular embodiment a compound of Formula (IXb),

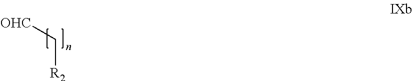

is used for the preparation of a compound of Formula (I).

In another particular embodiment a compound of Formula (Xa) or (Xb),

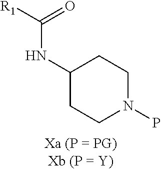

is used for the preparation of a compound of Formula (I).

In another particular embodiment a compound of Formula (XIa) or (XIb),

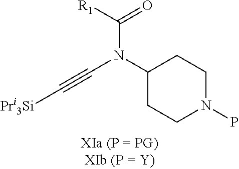

is used for the preparation of a compound of Formula (I).

In another particular embodiment a compound of Formula (XII),

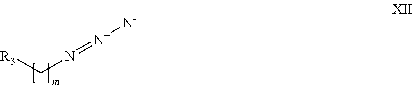

is used for the preparation of a compound of Formula (I).

The obtained reaction products may, if desired, be purified by conventional methods, such as crystallisation and chromatography. Where the above described processes for the preparation of compounds of the invention give rise to mixtures of stereoisomers, these isomers may be separated by conventional techniques such as preparative chromatography. If there are chiral centers the compounds may be prepared in racemic form, or individual enantiomers may be prepared either by enantiospecific synthesis or by resolution.

One preferred pharmaceutically acceptable form of a compound of the invention is the crystalline form, including such form in pharmaceutical composition. In the case of salts and also solvates of the compounds of the invention the additional ionic and solvent moieties must also be non-toxic. The compounds of the invention may present different polymorphic forms, it is intended that the invention encompasses all such forms.

Another aspect of the invention refers to a pharmaceutical composition which comprises a compound according to the invention as described above according to general formula I or a pharmaceutically acceptable salt or stereoisomer thereof, and a pharmaceutically acceptable carrier, adjuvant or vehicle. The present invention thus provides pharmaceutical compositions comprising a compound of this invention, or a pharmaceutically acceptable salt or stereoisomers thereof together with a pharmaceutically acceptable carrier, adjuvant, or vehicle, for administration to a patient.

Examples of pharmaceutical compositions include any solid (tablets, pills, capsules, granules etc.) or liquid (solutions, suspensions or emulsions) composition for oral, topical or parenteral administration.

In a preferred embodiment the pharmaceutical compositions are in oral form, either solid or liquid. Suitable dose forms for oral administration may be tablets, capsules, syrops or solutions and may contain conventional excipients known in the art such as binding agents, for example syrup, acacia, gelatin, sorbitol, tragacanth, or polyvinylpyrrolidone; fillers, for example lactose, sugar, maize starch, calcium phosphate, sorbitol or glycine; tabletting lubricants, for example magnesium stearate; disintegrants, for example starch, polyvinylpyrrolidone, sodium starch glycollate or microcrystalline cellulose; or pharmaceutically acceptable wetting agents such as sodium lauryl sulfate.

The solid oral compositions may be prepared by conventional methods of blending, filling or tabletting. Repeated blending operations may be used to distribute the active agent throughout those compositions employing large quantities of fillers. Such operations are conventional in the art. The tablets may for example be prepared by wet or dry granulation and optionally coated according to methods well known in normal pharmaceutical practice, in particular with an enteric coating.

The pharmaceutical compositions may also be adapted for parenteral administration, such as sterile solutions, suspensions or lyophilized products in the appropriate unit dosage form. Adequate excipients can be used, such as bulking agents, buffering agents or surfactants.

The mentioned formulations will be prepared using standard methods such as those described or referred to in the Spanish and US Pharmacopoeias and similar reference texts.

Administration of the compounds or compositions of the present invention may be by any suitable method, such as intravenous infusion, oral preparations, and intraperitoneal and intravenous administration. Oral administration is preferred because of the convenience for the patient and the chronic character of the diseases to be treated.

Generally an effective administered amount of a compound of the invention will depend on the relative efficacy of the compound chosen, the severity of the disorder being treated and the weight of the sufferer. However, active compounds will typically be administered once or more times a day for example 1, 2, 3 or 4 times daily, with typical total daily doses in the range of from 0.1 to 1000 mg/kg/day.

The compounds and compositions of this invention may be used with other drugs to provide a combination therapy. The other drugs may form part of the same composition, or be provided as a separate composition for administration at the same time or at different time.

Another aspect of the invention refers to the use of a compound of the invention or a pharmaceutically acceptable salt or isomer thereof in the manufacture of a medicament.

Another aspect of the invention refers to a compound of the invention according as described above according to general formula I, or a pharmaceutically acceptable salt or isomer thereof, for use as a medicament for the treatment of pain. Preferably the pain is medium to severe pain, visceral pain, chronic pain, cancer pain, migraine, inflammatory pain, acute pain or neuropathic pain, allodynia or hyperalgesia. This may include mechanical allodynia or thermal hyperalgesia.

Another aspect of the invention refers to the use of a compound of the invention in the manufacture of a medicament for the treatment or prophylaxis of pain.

In a preferred embodiment the pain is selected from medium to severe pain, visceral pain, chronic pain, cancer pain, migraine, inflammatory pain, acute pain or neuropathic pain, allodynia or hyperalgesia, also preferably including mechanical allodynia or thermal hyperalgesia.

Another aspect of this invention relates to a method of treating or preventing pain which method comprises administering to a patient in need of such a treatment a therapeutically effective amount of a compound as above defined or a pharmaceutical composition thereof. Among the pain syndromes that can be treated are medium to severe pain, visceral pain, chronic pain, cancer pain, migraine, inflammatory pain, acute pain or neuropathic pain, allodynia or hyperalgesia, whereas this could also include mechanical allodynia or thermal hyperalgesia.

The present invention is illustrated below with the aid of examples. These illustrations are given solely by way of example and do not limit the general spirit of the present invention.

### EXAMPLES

### General Experimental Part (Methods and Equipment of the Synthesis and Analysis

### Method Description

A process is described in Scheme 1 for the preparation of compounds of general formula I, wherein R₁, R₂, R₃, m, n and X have the meanings defined above.

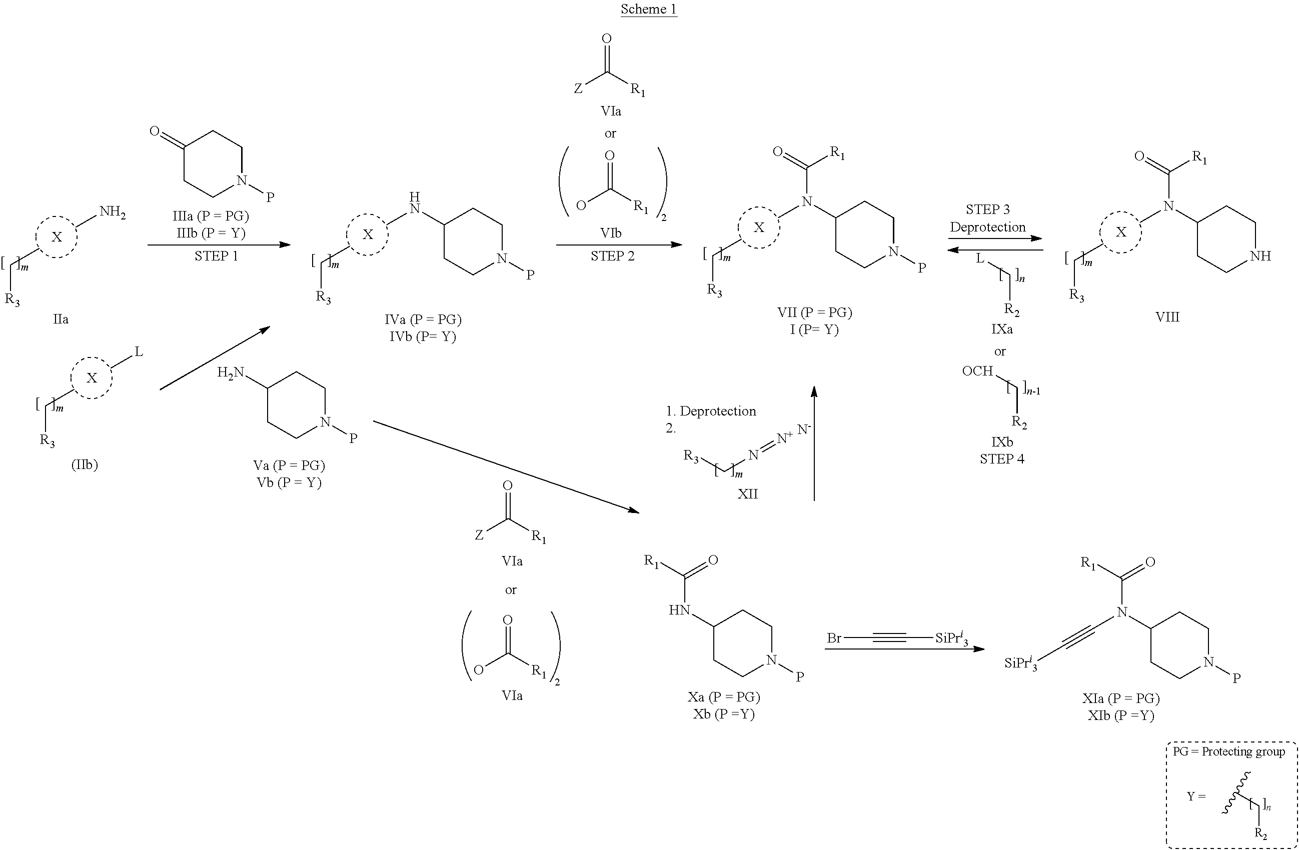

Where, L is a leaving group such as halogen, mesylate, tosylate or triflate and Z is chloro, bromo, methoxy or ethoxy, Y is the group indicated in a square in Scheme 1 and PG is a protecting group.

This process is carried out as described below:

Step 1: The compounds of formula IVa or IVb are prepared by reductive amination of compounds of formula IIa with a compound of formula IIIa or IIIb, in the presence of a reductive reagent, preferably sodium triacetoxyborohydride, in a suitable solvent, preferably dichloromethane, at a suitable temperature comprised between room temperature and the solvent reflux temperature, preferably at room temperature.

Alternatively, compounds of formula IVa or IVb can be obtained by reaction of piperidinylamino derivatives of formula Va or Vb with alkylating agents of formula IIb. The alkylation reaction is carried out in a suitable solvent, such as acetonitrile, dichloromethane, 1,4-dioxane or dimethylformamide, preferably in acetonitrile, in the presence of an inorganic base such as K₂CO₃ or Cs₂CO₃, or an organic base such as triethylamine or diisopropylethylamine, preferably diisopropylethylamine, at a suitable temperature comprised between room temperature and the solvent reflux temperature, preferably heating, or alternatively, the reactions can be carried out in a microwave reactor.

Step 2: Compounds of general formula VII or I are prepared by acylation of compounds of formula IVa or IVb with an acyl halide of formula VIa or with an anhydride of formula VIb. This reaction is carried out in the presence of a suitable solvent, such as acetonitrile, dichloromethane, 1,4-dioxane, 1,2-dicloroethane, toluene or dimethylformamide, in the presence of an organic base such as triethylamine, pyridine or diisopropylethylamine, at a suitable temperature comprised between room temperature and the solvent reflux temperature, or alternatively, the reactions can be carried out in a microwave reactor.

For compounds of general formula VII, wherein P is a protecting group, two additional steps are necessary to obtain compounds of formula I:

Step 3: A compound of formula VIII is prepared by deprotection of a compound of formula VII. If the protecting group is benzyl, the deprotection is carried out with hydrogen at a pressure comprised between 1 and 10 bar, in the presence of Pd, in a suitable solvent such as methanol or ethanol, optionally in the presence of an acid such as acetic or hydrochloric acid at a suitable temperature comprised between room temperature and the solvent reflux temperature, preferably at room temperature. If the protecting group is Boc, the deprotection is carried out in the presence of an acid such as HCl or trifluoroacetic acid, in a suitable solvent such as dichloromethane, at a suitable temperature comprised between room temperature and the solvent reflux temperature.

Step 4: From deprotected compounds of general formula VIII, compounds of general formula I can be prepared by reaction with suitable reagents, such as those of formula IXa-b, using different conditions depending on the reagent nature. Thus:

The alkylation reaction with a compound of formula IXa is carried out in a suitable solvent, such as acetonitrile, dichloromethane, 1,4-dioxane, ethanol or dimethylformamide, preferably in acetonitrile, in the presence of an inorganic base such as K₂CO₃ or Cs₂CO₃, or an organic base such as triethylamine or diisopropylethylamine, preferably K₂CO₃, at a suitable temperature comprised between room temperature and the solvent reflux temperature, preferably heating, or alternatively, this reaction can be carried out in a microwave reactor. Additionally, an activating agent such as NaI or KI can be used.

The reductive amination reaction between a compound of formula VIII and a compound of formula IXb is carried out in the presence of a reductive reagent, preferably sodium triacetoxyborohydride, in a protic solvent, preferably methanol at a suitable temperature, preferably room temperature. Alternatively the reaction can be carried out in an aprotic solvent, preferably tetrahydrofuran or dichloroethane, in the presence of an acid, preferably acetic acid.

Additionally, compounds of formula VIII or I wherein

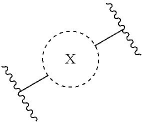

is triazole can be obtained in an alternative three step procedure from compounds of general formula Va or Vb. This process involves the acylation reaction between a compound of formula VIa or VIb and an amine of formula Va or Vb, under the reaction conditions previously described in step 2, to give amide derivatives of formula Xa or Xb.

Triisopropylsilylethynylamides of formula XIa or XIb are prepared by treating compounds of formula Xa or Xb with (bromoethynyl)triisopropylsilane, in the presence of 1.10-phenanthroline, a copper salt, preferably copper(II) sulfate pentahydrate and an inorganic base such as potassium phosphate or potassium hexamethylsilazane, preferably potassium phosphate, in a suitable solvent, such as toluene or 1,4-dioxane, preferably in toluene, at a suitable temperature comprised between room temperature and the solvent reflux temperature, preferably at the reflux temperature.

The final step of this alternative method involves deprotection of compounds of formula XIa or XIb by treatment with a fluoride reagent, such as tetrabutylammonium fluoride, in a suitable solvent, such as tetrahydrofuran and subsequent reaction of the alkyne intermediates with azide derivatives of formula XII to render compounds of general formula VII or I. This cyclization reaction is carried out in the presence of a copper catalyst, an organic base such as triethylamine or diisopropylethylamine, preferably diisopropylethylamine, in suitable solvent, such as tetrahydrofuran, at a suitable temperature comprised between room temperature and the solvent reflux temperature, preferably at room temperature.

The process described by Steps 1 to 4 and the corresponding alternative methods, represent the general route for the preparation of compounds of formula I. Additionally, the functional groups present in any of the positions can be interconverted using reactions known to those skilled in the art.

Compounds of formula IIa, IIb, IIIa, IIIb, Va, Vb, VIa, VIb, IXa, IXb and XII where R₁, R₂, R₃, m, n, L, X, Y and Z have the meanings as defined above, are commercially available or can be prepared by conventional methods described in the bibliography.

### Examples

### Intermediates and Examples

The following abbreviations are used in the examples:

### ACN: Acetonitrile

AcOH: Acetic acid

### Anh: Anhydrous

BINAP: 2,2′-Bis(diphenylphosphino)-1,1′-binaphthyl
Bn: Benzyl group

### Conc: Concentrated

### CH: Cyclohexane

### DCM: Dichloromethane

### DCE: 1,2-Dicloroethane

### DIPEA: N,N-Diisopropylethylamine

### DMF: Dimethylformamide

DMSO: Dimethyl sulfoxide
EDC: 1-Ethyl-3-(3-dimethylaminopropyl)carbodiimide

### EtOH: Ethanol

Et₂O: Diethyl ether
EtOAc: Ethyl acetate

### Ex: Example

### h: Hour/s

### HOBt: Hydroxybenzotriazole

HPLC: High-performance liquid chromatography

### INT: Intermediate

### MeOH: Methanol

MS: Mass spectrometry

### Min: Minutes

### Quant: Quantitative

### Ret: Retention

rt: Room temperature

### Sat: Saturated

### TEA: Et₃N, Triethylamine

TFA: Trifluoroacetic acid

### THF: Tetrahydrofuran

### Wt: Weight

The following methods were used to obtain the HPLC-MS data:

A: Column Acquity UPLC BEH C18 2.1×50 mm, 1.7 μm; flow rate 0.61 mL/min; A: NH₄HCO₃ 10 mM; B: ACN; Gradient: 0.3 min in 98% A, 98% A to 5% A in 2.52 min, 1.02 min in 5% A, 5% A to 98% A in 0.34 min, 0.57 min in 98% A

B: Column: Aqcuity BEH C18 2.1×50 mm 1.7 μm; flow rate 600 μl/min; A: NH₄HCO₃ 10 mM; B: ACN; Gradient: 0.3 min in 90% A, 90% A to 5% A in 2.7 min, 0.7 min in 5% A, 5% A to 90% A in 0.1 min, 1.2 min in 90% A
C: Column: SunFire C18, 3.5 μm, 2.1×50 mm; flow rate: 0.3 mL/min; A: CH₃CN:MeOH (1:1); B: Water; C: 100 mM ammonium acetate pH 7; Gradient: 2 min in 10:85:5+from 10:85:5 to 95:0:5 in 6 min+7 min in 95:0:5.
D: Column: SunFire C18, 4.6×50 mm, 3.5 μm; flow rate 0.3 mL/min; A (acetonitrile) and B (H₂O with 2% formic acid); Gradient: 5 min from 85:15 to 5:95.

### Intermediate 1A. 2-(4-Fluorophenyl)pyrimidin-5-amine

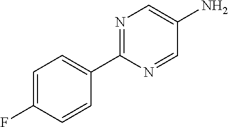 [SMILES: Nc1cnc(-c2ccc(F)cc2)nc1]

2-Chloropyrimidin-5-amine (500 mg, 3.86 mmol) was added to a suspension of an aqueous Na₂CO₃ solution (2.0 M, 3.9 mL, 7.72 mmol), Pd(dppf)Cl₂.CH₂Cl₂ (252 mg, 0.308 mmol) and 4-fluorophenyl boronic acid (801 mg, 5.78 mmol) in 1,4-dioxane (15 mL). The reaction mixture was refluxed for 5 h and allowed to reach rt, poured into water (20 mL) and extracted with EtOAc. The combined organic layers were dried over anh. Na₂SO₄, filtered and concentrated. The crude product thus obtained was purified by flash chromatography on silica gel, gradient acetone/hexane (40:0) to give the title compound as yellow oil (765 mg, 94% yield).

¹H-NMR (CDCl₃, 250 MHz, δ): 8.27 (m, 4H, ArH); 7.11 (m, 2H, ArH); 3.77 (bs, 2H, NH₂).

HPLC-MS (Method C): Ret, 7.84 min; ESI⁺-MS m/z, 190.2 (M+1).

This method was used for the preparation of intermediates 1B-C using suitable starting materials:

| | | | | | Ret | MS |
|---|---|---|---|---|---|---|
| INT | EST | Structure | Chemical name | Method | (min) | (M + H) |
| 1B | | 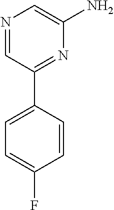 [SMILES: Nc1cncc(-c2ccc(F)cc2)n1] | 6-(4- fluorophenyl)pyrazin- 2-amine | C | 8.00 | 190.2 |
| 1C | | 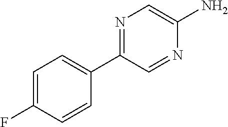 [SMILES: Nc1cnc(-c2ccc(F)cc2)cn1] | 5-(4- fluorophenyl)pyrazin- 2-amine | C | 8.00 | 190.2 |

### Intermediate 1D. 1H-Indazol-3-amine

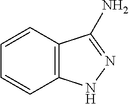

### a) 2-(2-(Diphenylmethylene)hydrazinyl)benzonitrile

(Diphenylmethylene)hydrazine (7.1 g, 36.26 mmol), Pd(OAc)₂ (0.32 g, 1.65 mmol) and BINAP (1.13 g, 1.81 mmol) were added to a Raddley tube, under nitrogen, and dissolved in toluene (49 mL). The mixture was heated at 100° C. for 15 min and then it was cooled down to rt. 2-Bromobenzonitrile (6.00 g, 32.96 mmol), Cs₂CO₃ (15.03 g, 46.14 mmol) and toluene (17 mL) were added under nitrogen and the reaction mixture was stirred at 100° C. for 43 h. The mixture was filtered off over a pad of Celite®, washed with DCM and the filtrate concentrated to dryness. The crude product thus obtained was purified by column chromatography on silica (5% EtOAc/hexane) to give the title compound as green solid (7.2 g, 74% yield).

RMN-¹H (CDCl₃, 250 MHz, δ): 8.18 (s, 1H, NH); 7.84-7.30 (m, 13H, ArH); 6.83 (t, J=7.4 Hz, 1H, ArH).

### b) Title Compound

Over a suspension of 2-(2-(diphenylmethylene)hydrazinyl)benzonitrile obtained in the previous step (7.2 g, 24.25 mmol) in MeOH (50 mL), p-toluenesulfonic acid monohydrate (9.20 g, 48.36 mmol) was added and the mixture was stirred at reflux for 15 h. The solvent was concentrated off. The crude residue was diluted with a sat. aqueous K₂CO₃ solution (100 mL) and extracted with EtOAc (150 mL). The combined organic layers were washed with sat NaCl solution, dried over Na₂SO₄, filtered and concentrated. The solid thus obtained was triturated with hexane (30 mL) at 40° C., filtered and washed with hexane to give the title compound as cream solid (2.98 g, 93% yield)

RMN-¹H (MeOD, 250 MHz, δ): 7.67 (dt, J=8.2, 0.9 Hz, 1H, ArH); 7.35-7.25 (m, 2H, ArH); 7.02-6.95 (m, 1H, ArH).

### Intermediate 2A. N-(1-benzylpiperidin-4-yl)-2-(4-fluorophenyl)pyrimidin-5-amine

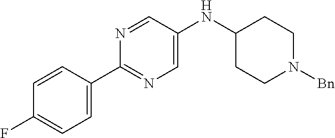 [SMILES: Fc1ccc(-c2ncc(NC3CCN(Cc4ccccc4)CC3)cn2)cc1]

1-Benzylpiperidin-4-one (1.33 mL, 7.2 mmol) was added to a suspension of NaBH(OAc)₃ (1.52 g, 7.2 mmol), AcOH (0.617 mL, 10.8 mmol) and 2-(4-fluorophenyl)pyrimidin-5-amine (INT 1A, 0.682 g, 3.60 mmol) in DCM (30 mL). The reaction mixture was stirred at rt for 24 h. Then, additional 1-benzylpiperidin-4-one (0.665 g, 3.6 mmol), NaBH(OAc)₃ (0.76 g, 3.6 mmol) and AcOH (0.308 mL, 5.4 mmol) were added and the mixture was stirred at rt for 15 h. The mixture was quenched with sat aqueous NH₄Cl solution (30 mL) and extracted with DCM. The combined organic layers were dried over anh. Na₂SO₄, filtered and concentrated. The crude product thus obtained was purified by flash chromatography on silica gel (2-7% MeOH/DCM) to give the title compound as white solid (865 mg, 67% yield).

¹H-NMR (CDCl₃, 250 MHz, δ): 8.25 (m, 2H, ArH); 8.16 (s, 2H, ArH); 7.40-7.29 (m, 5H, ArH); 7.1 (m, 2H, ArH); 3.68 (bs, 1H, NH); 3.59 (s, 2H, CH₂); 3.37 (m, 1H, CH); 2.93 (m, 2H, CH₂); 2.32-1.98 (m, 4H, CH₂); 1.60 (m, 2H, CH₂).

HPLC-MS (Method C): Ret, 10.2 min; ESI⁺-MS m/z, 363.4 (M+1).

This method was used for the preparation of intermediates 2B-D using suitable starting materials:

| | | | | Ret | MS |
|---|---|---|---|---|---|
| INT | Structure | Chemical name | Method | (min) | (M + H) |
| 2B | 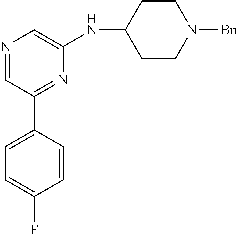 [SMILES: Fc1ccc(-c2cncc(NC3CCN(Cc4ccccc4)CC3)n2)cc1] | N-(1- benzylpiperidin-4- yl)-6-(4- fluorophenyl)pyrazin- 2-amine | C | 10.4 | 363.4 |
| 2C | 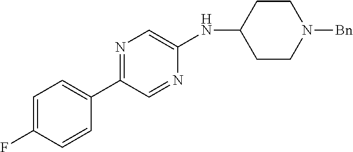 [SMILES: Fc1ccc(-c2cnc(NC3CCN(Cc4ccccc4)CC3)cn2)cc1] | N-(1- benzylpiperidin-4- yl)-5-(4- fluorophenyl)pyrazin- 2-amine | C | 10.2 | 362.9 |
| 2D | 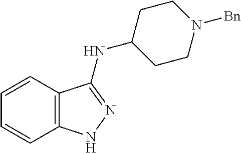 [SMILES: c1ccc(CN2CCC(Nc3n[nH]c4ccccc34)CC2)cc1] | N-(1- benzylpiperidin-4- yl)-1H-indazol-3- amine | C | 10.8 | 307.4 |

### Intermediate 2E. N-(1-benzylpiperidin-4-yl)-5-phenyl-1,3,4-oxadiazol-2-amine

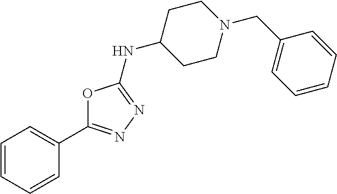 [SMILES: c1ccc(CN2CCC(Nc3nnc(-c4ccccc4)o3)CC2)cc1]

A mixture of 5-phenyl-1,3,4-oxadiazol-2-ol (0.3 g, 1.85 mmol), 1-benzylpiperidin-4-amine (0.7 g, 3.7 mmol), (1H-benzo[d][1,2,3]triazol-1-yloxy) tris(dimethylamino)phosphonium hexafluorophosphate(V) (0.9 g, 2 mmol) and DIPEA (0.48 g, 3.7 mmol) was dissolved in DMF (5 mL) at rt for 1 h. The mixture was concentrated and the residue was treated with EtOAc and brine. The phases were separated and the organic phase was dried with anh. Na₂SO₄, filtered and concentrated. The crude product thus obtained was purified by flash chromatography on silica gel, gradient Et₂O/EtOAc (1:1) to give the title compound (207 mg, 34% yield).

HPLC-MS (Method A): Ret, 1.79 min; ESI⁺-MS m/z, 335.2 (M+1).

### Intermediate 2F. N-(1-Benzylpiperidin-4-yl)-3-phenyl-1,2,4-thiadiazol-5-amine

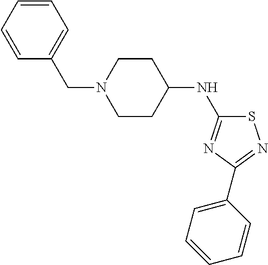 [SMILES: c1ccc(CN2CCC(Nc3nc(-c4ccccc4)ns3)CC2)cc1]

5-Chloro-3-phenyl-1,2,4-thiadiazole (340 mg, 1.7 mmol) was added to a solution of 1-benzylpiperidin-4-amine (494 mg, 2.6 mmol) and N-ethyl-N-isopropylpropan-2-amine (592 μL, 3.46 mmol) in ACN (4 mL) in a vial under nitrogen atmosphere. The reaction mixture was heated under microwave irradiating conditions for 2.5 h at 190° C. The solvent was evaporated and the residue was dissolved in EtOAc and water. The aqueous layer was extracted with EtOAc and the combined organic layers were dried over anh. Na₂SO₄, filtered and concentrated. The crude product thus obtained was purified by flash chromatography on gold silica gel, gradient CH/EtOAc to give the title compound as yellow solid (470 mg, 77% yield).

HPLC-MS (Method A): Ret, 2.29 min; ESI⁺-MS m/z, 351.3 (M+1).

This method was used for the preparation of intermediates 2G-I using the required halides.

| | | | | Ret | MS |
|---|---|---|---|---|---|
| INT | Structure | Chemical name | Method | (min) | (M + H) |
| 2G | 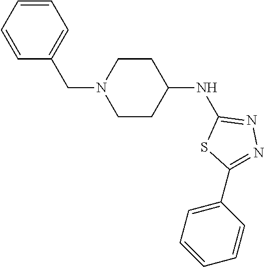 [SMILES: c1ccc(CN2CCC(Nc3nnc(-c4ccccc4)s3)CC2)cc1] | N-(1- benzylpiperidin-4- yl)-5-phenyl-1,3,4- thiadiazol-2-amine | A | 1.97 | 351.3 |
| 2H | 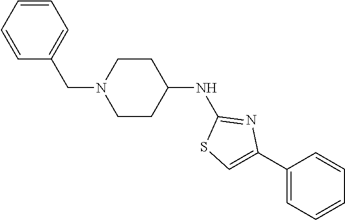 [SMILES: c1ccc(CN2CCC(Nc3nc(-c4ccccc4)cs3)CC2)cc1] | N-(1- benzylpiperidin-4- yl)-4- phenylthiazol-2- amine | B | 3.36 | 350.3 |
| 2I | 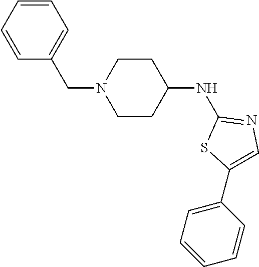 [SMILES: c1ccc(CN2CCC(Nc3ncc(-c4ccccc4)s3)CC2)cc1] | N-(1- benzylpiperidin-4- yl)-5- phenylthiazol-2- amine | A | 2.25 | 350.2 |

### Intermediate 2J. N-(1-Benzylpiperidin-4-yl)propionamide

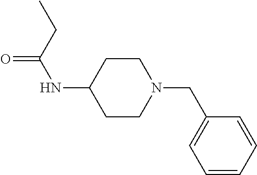 [SMILES: CCC(=O)NC1CCN(Cc2ccccc2)CC1]

Propionyl chloride (4 mL, 46.2 mmol) was added to a solution of 1-benzylpiperidin-4-amine (8 g, 42 mmol) and DIPEA (10.8 mL, 63 mmol) in DCM. The reaction mixture was stirred at rt overnight. The mixture was quenched with sat. aqueous NaHCO₃ solution and extracted with DCM. The combined organic layers were dried over anh. Na₂SO₄, filtered and concentrated. The crude product thus obtained was purified by flash chromatography on silica gel, DCM/MeOH (9:1) to give the title compound (5.7 g, 55% yield).

HPLC-MS (Method A): Ret, 1.33 min; ESI⁺-MS m/z, 247.2 (M+1).

This method was used for the preparation of intermediates 2K-L using suitable starting materials:

| | | | | Ret | MS |
|---|---|---|---|---|---|
| INT | Structure | Chemical name | Method | (min) | (M + H) |
| 2K | 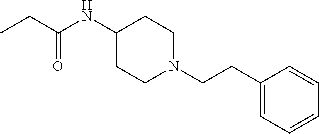 [SMILES: CCC(=O)NC1CCN(CCc2ccccc2)CC1] | N-(1- phenethylpiperidin- 4-yl)propionamide | A | 1.38 | 261.3 |
| 2L | 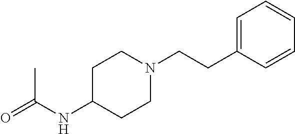 [SMILES: CC(=O)NC1CCN(CCc2ccccc2)CC1] | N-(1- phenethylpiperidin- 4-yl)acetamide | A | 1.28 | 247.2 |

### Intermediate 3A. N-(1-Benzylpiperidin-4-yl)-N-((triisopropylsilyl)ethynyl)propionamide

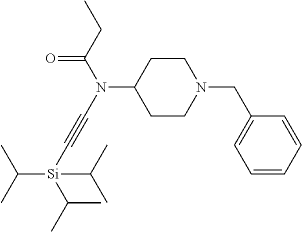 [SMILES: CCC(=O)N(C#C[Si](C(C)C)(C(C)C)C(C)C)C1CCN(Cc2ccccc2)CC1]

A mixture of N-(1-benzylpiperidin-4-yl)propionamide (INT 2J, 8 g, 32.3 mmol), copper(II) sulfate pentahydrate (2 g, 1.7 mmol), tripotassium phosphate (13.7 g, 65 mmol) and 1.10-phenanthroline (1.17 g, 6.5 mmol) was dissolved in toluene (75 mL) at rt. Then, (bromoethynyl)triisopropylsilane (9.3 g, 35.6 mmol) was added and the mixture was heated at 110° C. for 2 days. The mixture was concentrated and the residue thus obtained was treated with EtOAc and brine, dried with anh. Na₂SO₄, filtered and concentrated. The crude product thus obtained was purified by flash chromatography on silica gel, gradient CH/EtOAc from (1:0) to (9:1) to give the title compound (1.9 g, 14% yield).

HPLC-MS (Method A): Ret, 3.08 min; ESI⁺-MS m/z, 427.4 (M+1).

This method was used for the preparation of intermediates 3-C using suitable starting materials:

| | | | | Ret | MS |
|---|---|---|---|---|---|
| INT | Structure | Chemical name | Method | (min) | (M + H) |
| 3B | 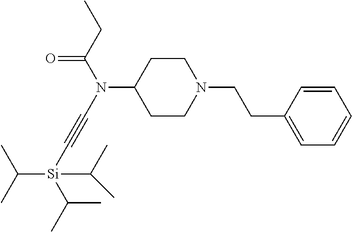 [SMILES: CCC(=O)N(C#C[Si](C(C)C)(C(C)C)C(C)C)C1CCN(CCc2ccccc2)CC1] | N-(1- phenethylpiperidin- 4-yl)-N- ((triisopropylsilyl) ethynyl)propionamide | A | 3.01 | 441.4 |
| 3C | 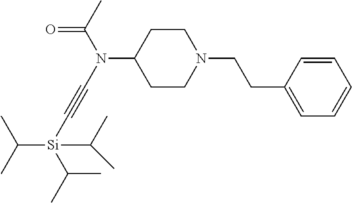 [SMILES: CC(=O)N(C#C[Si](C(C)C)(C(C)C)C(C)C)C1CCN(CCc2ccccc2)CC1] | N-(1- phenethylpiperidin- 4-yl)-N- ((triisopropylsilyl) ethynyl)acetamide | A | 2.75 | 427.4 |

### Intermediate 4. N-(1-Benzylpiperidin-4-yl)-N-(1H-indazol-3-yl)propionamide

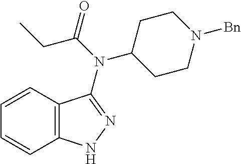 [SMILES: CCC(=O)N(c1n[nH]c2ccccc12)C1CCN(Cc2ccccc2)CC1]

### a) N-(1-Benzylpiperidin-4-yl)-N-(1-propionyl-1H-indazol-3-yl)propionamide

The title compound was obtained following the procedure described in example 1 and using N-(1-benzylpiperidin-4-yl)-1H-indazol-3-amine (INT 2D, 6.7 g, 21.89 mmol) as starting material.

### b) Title Compound

Over a solution of N-(1-benzylpiperidin-4-yl)-N-(1-propionyl-1H-indazol-3-yl)propionamide obtained in the previous step (21.89 mmol) in THF/MeOH (1:1, 100 mL), aqueous LiOH solution (2M, 16.4 mL, 32.83 mmol) was added and the mixture was stirred at rt for 15 h. The solvent was removed and the crude residue was diluted with aqueous NaOH (10%, 100 mL) and extracted with DCM (150 mL). The combined organic layers were dried over Na₂SO₄, filtered and concentrated. The crude product thus obtained was purified by column chromatography on silica (40% acetone/hexane) to give the title compound as off white solid (3.26 g, 41% yield over two steps).

HPLC-MS (Method C): Ret, 16.89 min; ESI⁺-MS m/z, 363.5 (M+1).

### Example 1. N-(1-Benzylpiperidin-4-yl)-N-(2-(4-fluorophenyl)pyrimidin-5-yl)propionamide

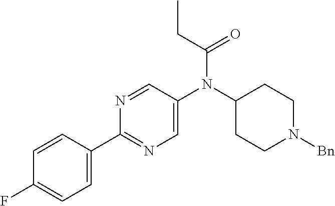 [SMILES: CCC(=O)N(c1cnc(-c2ccc(F)cc2)nc1)C1CCN(Cc2ccccc2)CC1]

Propionyl chloride (0.41 mL, 4.74 mmol) was added to a solution of N-(1-benzylpiperidin-4-yl)-2-(4-fluorophenyl)pyrimidin-5-amine (INT 2A, 860 mg, 2.37 mmol) and DIPEA (0.811 mL, 4.74 mmol) in DCM (15 mL). The reaction mixture was stirred at reflux for 3.5 h and allowed to reach rt. The mixture was quenched with sat. aqueous NaHCO₃ solution (20 mL) and extracted with DCM. The combined organic layers were dried over anh. Na₂SO₄, filtered and concentrated. The crude product thus obtained was purified by flash chromatography on silica gel, (20-40% EtOAc/hexane) to give the title compound as a white solid (831 mg, 84% yield).

HPLC-MS (Method C): Ret, 20.44 min; ESI⁺-MS m/z, 419.5 (M+1).

This method was used for the preparation of examples 2-4 using suitable starting materials:

| | | | | Ret | MS |
|---|---|---|---|---|---|
| INT | Structure | Chemical name | Method | (min) | (M + H) |
| 2 | 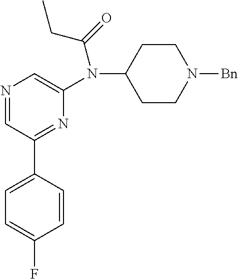 [SMILES: CCC(=O)N(c1cncc(-c2ccc(F)cc2)n1)C1CCN(Cc2ccccc2)CC1] | N-(1- benzylpiperidin-4- yl)-N-(6-(4- fluorophenyl)pyrazin- 2- yl)propionamide | C | 19.68 | 419.5 |
| 3 | 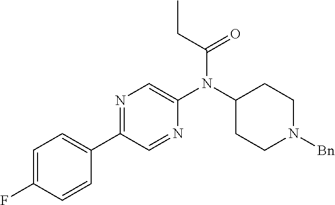 [SMILES: CCC(=O)N(c1cnc(-c2ccc(F)cc2)cn1)C1CCN(Cc2ccccc2)CC1] | N-(1- benzylpiperidin-4- yl)-N-(5-(4- fluorophenyl)pyrazin- 2- yl)propionamide | C | 19.9 | 419.5 |
| 4 | 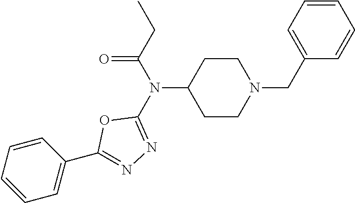 [SMILES: CCC(=O)N(c1nnc(-c2ccccc2)o1)C1CCN(Cc2ccccc2)CC1] | N-(1- benzylpiperidin-4- yl)-N-(5-phenyl- 1,3,4-oxadiazol-2- yl)propionamide | A | 2.30 | 391.3 |

### Example 6. N-(1-Benzylpiperidin-4-yl)-N-(3-phenyl-1,2,4 thiadiazol-yl)propionamide

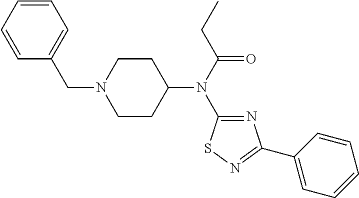 [SMILES: CCC(=O)N(c1nc(-c2ccccc2)ns1)C1CCN(Cc2ccccc2)CC1]

Propionic anhydride (0.234 mL, 1.82 mmol) was added to a solution of N-(1-benzylpiperidin-4-yl)-3-phenyl-1,2,4-thiadiazol-5-amine (INT 2F, 320 mg, 0.91 mmol), DIPEA (0.391 mL, 2.28 mmol) and catalytic amount of DMAP (11 mg, 0.09 mmol) in anh. DMF. The reaction mixture was stirred at 110° C. for 18 h and allowed to reach rt. The same amount of DIPEA and propionic anhydride was added and heated for 2 h more. The mixture was diluted with EtOAc and washed with an aqueous sat. NaHCO₃ solution and water. The organic layers were dried over anh. Na₂SO₄, filtered and concentrated. The crude product thus obtained was purified by flash chromatography on gold silica gel, (gradient CH/EtOAc) to give the title compound as a white solid (154 mg, 41% yield).

HPLC-MS (Method A): Ret, 2.64 min; ESI⁺-MS m/z, 407.3 (M+1).

This method was used for the preparation of examples 6-9 using suitable starting materials:

| | | | | Ret | MS |
|---|---|---|---|---|---|
| EX | Structure | Chemical name | Method | (min) | (M + H) |
| 6 | 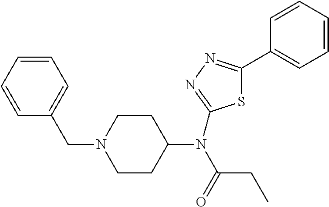 [SMILES: CCC(=O)N(c1nnc(-c2ccccc2)s1)C1CCN(Cc2ccccc2)CC1] | N-(1- benzylpiperidin-4- yl)-N-(5-phenyl- 1,3,4-thiadiazol-2- yl)propionamide | A | 2.37 | 407.3 |
| 7 | 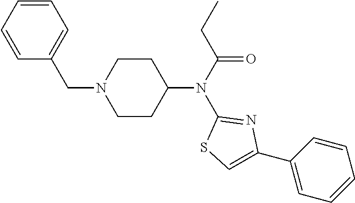 [SMILES: CCC(=O)N(c1nc(-c2ccccc2)cs1)C1CCN(Cc2ccccc2)CC1] | N-(1- benzylpiperidin-4- yl)-N-(4- phenylthiazol-2- yl)propionamide | A | 2.58 | 406.3 |
| 8 | 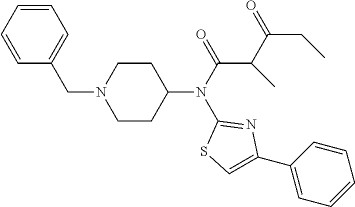 [SMILES: CCC(=O)C(C)C(=O)N(c1nc(-c2ccccc2)cs1)C1CCN(Cc2ccccc2)CC1] | N-(1- benzylpiperidin-4- yl)-2-methyl-3- oxo-N-(4- phenylthiazol-2- yl)pentanamide | A | 2.55 | 462.3 |
| 9 | 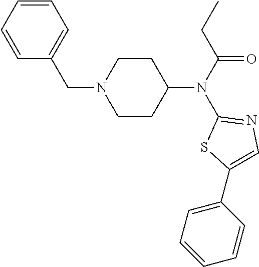 [SMILES: CCC(=O)N(c1ncc(-c2ccccc2)s1)C1CCN(Cc2ccccc2)CC1] | N-(1- benzylpiperidin-4- yl)-N-(5- phenylthiazol-2- yl)propionamide | A | 2.53 | 406.3 |

### Example 10. N-(2-(4-Fluorophenyl)pyrimidin-5-yl)-N-(1-phenethylpiperidin-4-yl)propionamide

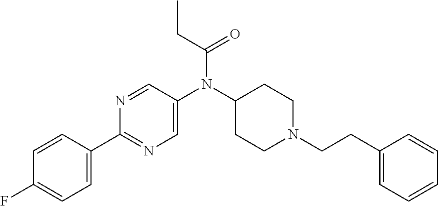 [SMILES: CCC(=O)N(c1cnc(-c2ccc(F)cc2)nc1)C1CCN(CCc2ccccc2)CC1]

### a) N-(2-(4-Fluorophenyl)pyrimidin-5-yl)-N-(piperidin-4-yl)propionamide

N-(1-Benzylpiperidin-4-yl)-N-(2-(4-fluorophenyl)pyrimidin-5-yl)propionamide (Example 1, 762 mg, 1.82 mmol) was added to a suspension of Pd(OH)₂ (255 mg, 18% Pd, 50% H₂O w/w, 0.18 mmol) and AcOH (10 μL, 0.182 mmol) in MeOH (20 mL). The suspension was stirred at rt under 1 bar of H₂ overnight. The reaction mixture was filtered through celite, washed with MeOH and concentrated, to give the title compound as yellow solid (679 mg, 598 mg theoretical weight, quant yield), that was used in the following step without further purification.

HPLC-MS (Method C): Ret, 8.22 min; ESI⁺-MS m/z, 329 (M+1).

### b) Title Compound

(2-Bromoethyl)benzene (0.133 mL, 0.989 mmol) was added to a suspension of N-(2-(4-fluorophenyl)pyrimidin-5-yl)-N-(piperidin-4-yl)propionamide obtained in step a (250 mg, 0.761 mmol) and K₂CO₃ (210 mg, 1.52 mmol) in ACN (10 mL). The reaction mixture was stirred at rt for 24 h, and additional (2-bromoethyl)benzene (51 μL, 0.38 mmol) was added and the mixture refluxed for 8 h. The reaction mixture was allowed to reach rt, poured into water (20 mL) and extracted with EtOAc. The combined organic layers were dried over anh. Na₂SO₄, filtered and concentrated. The crude product thus obtained was purified by column chromatography on silica (4% MeOH/DCM) to give the title compound as off white solid (193 mg, 49% yield, two steps).

HPLC-MS (Method C): Ret, 19.81 min; ESI⁺-MS m/z, 433.4 (M+1).

This method was used for the preparation of examples 11-12 using suitable starting materials:

| | | | | Ret | MS |
|---|---|---|---|---|---|
| Ex | Structure | Chemical name | Method | (min) | (M + H) |
| 11 | 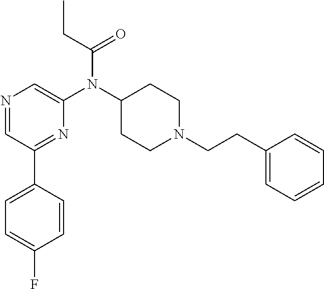 [SMILES: CCC(=O)N(c1cncc(-c2ccc(F)cc2)n1)C1CCN(CCc2ccccc2)CC1] | N-(6-(4- fluorophenyl)pyrazin- 2-yl)-N-(1- phenethylpiperidin- 4-yl)propionamide | C | 19.2 | 433.4 |
| 12 | 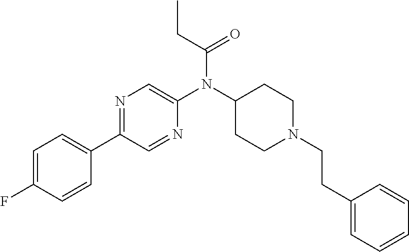 [SMILES: CCC(=O)N(c1cnc(-c2ccc(F)cc2)cn1)C1CCN(CCc2ccccc2)CC1] | N-(5-(4- fluorophenyl)pyrazin- 2-yl)-N-(1- phenethylpiperidin- 4-yl)propionamide | C | 19.26 | 433.4 |

### Example 13. N-(1-Benzylpiperidin-4-yl)-N-(1-phenyl-1H-indazol-3-yl)propionamide

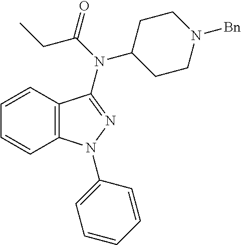 [SMILES: CCC(=O)N(c1nn(-c2ccccc2)c2ccccc12)C1CCN(Cc2ccccc2)CC1]

N-(1-Benzylpiperidin-4-yl)-N-(1H-indazol-3-yl)propionamide (INT 13, 127 mg, 0.35 mmol) was added to a suspension of phenylboronic acid (86 mg, 0.70 mmol), pyridine (0.056 mL, 0.70 mmol) and cupric acetate (95 mg, 0.52 mmol) in DCM (6 mL). The reaction mixture was stirred at rt for 24 h, filtered through cotton and washed with DCM (10 mL). The filtrate was washed with a sat. aqueous NH₄Cl solution and the organic phase was dried over anh. Na₂SO₄, filtered and concentrated to dryness. The crude product thus obtained was purified by flash chromatography on silica (20% acetone/hexane) and the resulting solid was triturated with hexane at −78° C., filtered and washed with hexane to give the title compound as white solid (65 mg, 42% yield).

HPLC-MS (Method C): Ret, 21.3 min; ESI⁺-MS m/z, 439.5 (M+1).

This method was used for the preparation of examples 14-17 using suitable starting materials:

| | | | | Ret | MS |
|---|---|---|---|---|---|
| Ex | Structure | Chemical name | Method | (min) | (M + H) |
| 14 | 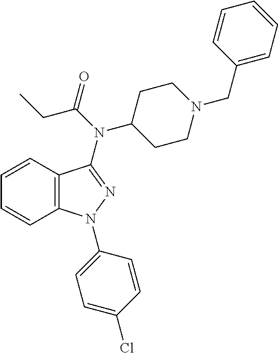 [SMILES: CCC(=O)N(c1nn(-c2ccc(Cl)cc2)c2ccccc12)C1CCN(Cc2ccccc2)CC1] | N-(1- benzylpiperidin- 4-yl)-N-(1-(4- chlorophenyl)- 1H-indazol-3- yl)propionamide | C | 22.22 | 473.2 |
| 15 | 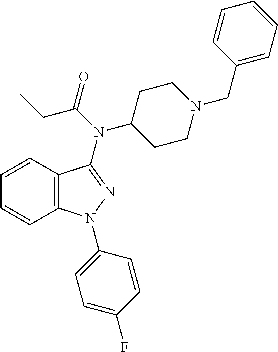 [SMILES: CCC(=O)N(c1nn(-c2ccc(F)cc2)c2ccccc12)C1CCN(Cc2ccccc2)CC1] | N-(1- benzylpiperidin- 4-yl)-N-(1-(4- fluorophenyl)- 1H-indazol-3- yl)propionamide | C | 21.27 | 457.3 |
| 16 | 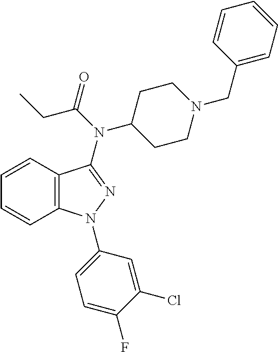 [SMILES: CCC(=O)N(c1nn(-c2ccc(F)c(Cl)c2)c2ccccc12)C1CCN(Cc2ccccc2)CC1] | N-(1- benzylpiperidin- 4-yl)-N-(1-(3- chloro-4- fluorophenyl)- 1H-indazol-3- yl)propionamide | C | 22.31 | 491.2 |
| 17 | 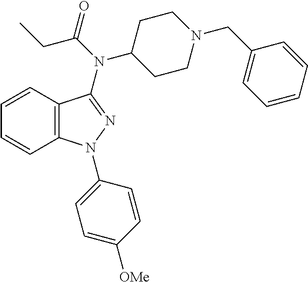 [SMILES: CCC(=O)N(c1nn(-c2ccc(OC)cc2)c2ccccc12)C1CCN(Cc2ccccc2)CC1] | N-(1- benzylpiperidin- 4-yl)-N-(1-(4- methoxyphenyl)- 1H-indazol-3- yl)propionamide | C | 20.31 | 469.5 |

### Example 18. N-(1-Benzylpiperidin-4-yl)-N-(1-(4-hydroxyphenyl)-1H-indazol-3-yl)propionamide

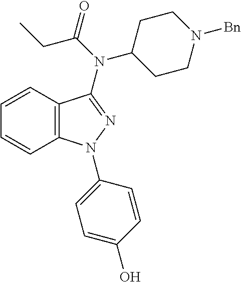 [SMILES: CCC(=O)N(c1nn(-c2ccc(O)cc2)c2ccccc12)C1CCN(Cc2ccccc2)CC1]

A solution of tribromoborane in DCM (1M, 0.9 mL, 0.91 mmol) was added to a solution of N-(1-benzylpiperidin-4-yl)-N-(1-(4-methoxyphenyl)-1H-indazol-3-yl)propionamide (Example 17, 85 mg, 0.18 mmol) in DCM (5 mL) cooled to −78° C. The mixture was allowed to reach rt and stirred for 14 h. The solvent was concentrated off and the crude residue was diluted with water, sat. aqueous NaHCO₃ solution until pH=8, and extracted with EtOAc. The combined organic layers were dried over Na₂SO₄, filtered and concentrated. The crude product thus obtained was purified by flash chromatography on silica (20-40% acetone/hexane) to give the title compound as cream solid (86 mg, 68% yield)

HPLC-MS (Method C): Ret, 19.37 min; ESI⁺-MS m/z, 455.1 (M+1).

### Example 19. N-(1-Benzylpiperidin-4-yl)-N-(1-(4-fluorophenyl)-1H-1,2,3-triazol-4-yl)propionamide

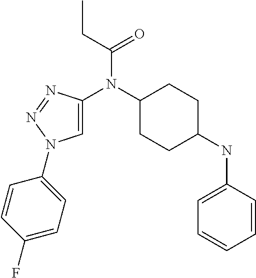 [SMILES: CCC(=O)N(c1cn(-c2ccc(F)cc2)nn1)C1CCC(Nc2ccccc2)CC1]

In a Schlenk tube under argon atmosphere, a solution of 1 N-(1-benzylpiperidin-4-yl)-N-((triisopropylsilyl)ethynyl)propionamide (INT 3A, 180 mg, 0.42 mmol) in THF, tetrabutylammonium fluoride trihydrate (1 M, 421 μL, 0.42 mmol) was added and the mixture was stirred 10 min at rt. Then, 1-azido-4-fluorobenzene (0.5 M, 928 μL, 0.464 mmol), CuI (40 mg, 0.211 mmol) and DIPEA (72 μL, 0.422 mmol) were added. The reaction mixture was stirred at 0° C. for 2 h. The mixture was allowed to reach rt and the solvent was evaporated. The crude product thus obtained was purified by column chromatography on silica (8:2, CH/EtOAc) to give the title compound (98 mg, 57% yield over two steps).

HPLC-MS (Method A): Ret 2.03 min; ESI⁺-MS n/z, 408.1 (M+1).

This method was used for the preparation of examples 20-25 using suitable starting materials:

| | | | | Ret | MS |
|---|---|---|---|---|---|
| Ex | Structure | Chemical name | Method | (min) | (M + H) |
| 20 | 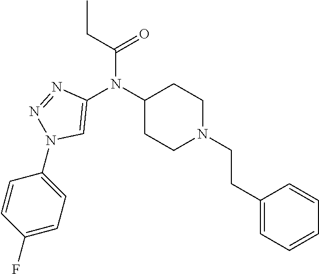 [SMILES: CCC(=O)N(c1cn(-c2ccc(F)cc2)nn1)C1CCN(CCc2ccccc2)CC1] | N-(1-(4- fluorophenyl)-1H- 1,2,3-triazol-4-yl)- N-(1- phenethylpiperidin- 4-yl)propionamide | A | 2.00 | 422.2 |
| 21 | 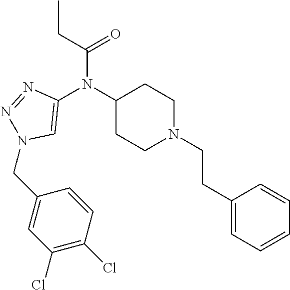 [SMILES: CCC(=O)N(c1cn(Cc2ccc(Cl)c(Cl)c2)nn1)C1CCN(CCc2ccccc2)CC1] | N-(1-(3,4- dichlorobenzyl)- 1H-1,2,3-triazol-4- yl)-N-(1- phenethylpiperidin- 4-yl)propionamide | A | 2.19 | 486 |
| 22 | 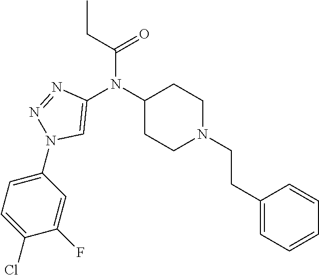 [SMILES: CCC(=O)N(c1cn(-c2ccc(Cl)c(F)c2)nn1)C1CCN(CCc2ccccc2)CC1] | N-(1-(4-chloro-3- fluorophenyl)-1H- 1,2,3-triazol-4-yl)- N-(1- phenethylpiperidin- 4-yl)propionamide | A | 2.21 | 456.1 |
| 23 | 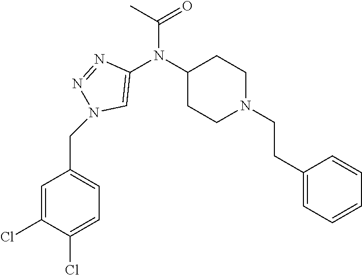 [SMILES: CC(=O)N(c1cn(Cc2ccc(Cl)c(Cl)c2)nn1)C1CCN(CCc2ccccc2)CC1] | N-(1-(3,4- dichlorobenzyl)- 1H-1,2,3-triazol-4- yl)-N-(1- phenethylpiperidin- 4-yl)acetamide | A | 2.90 | 472 |
| 24 | 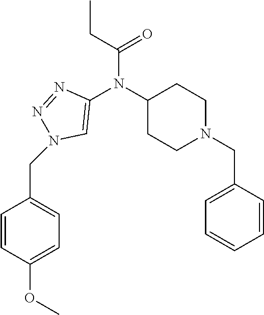 [SMILES: CCC(=O)N(c1cn(Cc2ccc(OC)cc2)nn1)C1CCN(Cc2ccccc2)CC1] | N-(1- benzylpiperidin-4- yl)-N-(1-(4- methoxybenzyl)- 1H-1,2,3-triazol-4- yl)propionamide | A | 1.93 | 434.4 |
| 25 | 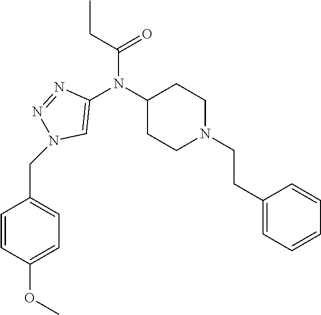 [SMILES: CCC(=O)N(c1cn(Cc2ccc(OC)cc2)nn1)C1CCN(CCc2ccccc2)CC1] | N-(1-(4- methoxybenzyl)- 1H-1,2,3-triazol-4- yl)-N-(1- phenethyl)piperidin- 4-yl)propionamide | A | 1.91 | 448.4 |

### Example 26. N-(1-(4-Fluorophenyl)-1H-1,2,3-triazol-5-yl)-N-(1-phenethylpiperidin-4-yl)propionamide

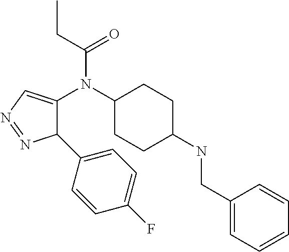 [SMILES: CCC(=O)N(C1=CN=NC1c1ccc(F)cc1)C1CCC(NCc2ccccc2)CC1]

### a) N-Ethynyl-N-(1-phenethylpiperidin-4-yl)propionamide

In a Schlenk tube under argon atmosphere, a solution of N-(1-phenethylpiperidin-4-yl)-N-((triisopropylsilyl)ethynyl)propionamide (INT 3B, 155 mg, 0.352 mmol) in THF was charged, tetrabutylammonium fluoride trihydrate (1M, 352 μL, 0.35 mmol) was added and the mixture was stirred 10 min at rt. The mixture was quenched with sat. aqueous NaHCO₃ solution and extracted with EtOAc. The combined organic layers were dried over anh Na₂SO₄, filtered and concentrated to afford the title compound

### b) Title Compound

The compound obtained in the previous step was (0.352 mmol), charged in a Schlenk tube under argon atmosphere and Cp*RuCl(PPh₃)₂ (cat. 5% mol) and 1-azido-4-fluorobenzene (0.5 M, 774 μL, 0.387 mmol) were added, purged with argon, and backfilled for three times. Dry toluene (8 mL) was added and the reaction was stirred at 80° C. for 25 h. Reaction mixture was then cooled down to rt, quenched with water and extracted with EtOAc. The combined organic layers were dried over Na₂SO₄, filtered and concentrated. The crude product thus obtained was purified by column chromatography on silica (CH/EtOAc 40%) to give the title compound (15 mg, 10% yield, two steps).

HPLC-MS (Method A): Ret 2.04 min; ESI⁺-MS n/z, 422.3 (M+1).

### Example 27. N-(2-(4-Fluorophenyl)-2H-1,2,3-triazol-4-yl)-N-(1-phenethylpiperidin-4-yl)propionamide

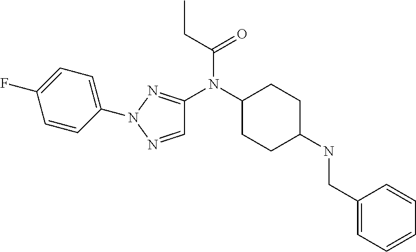 [SMILES: CCC(=O)N(c1cnn(-c2ccc(F)cc2)n1)C1CCC(NCc2ccccc2)CC1]

### a) N-(1-Phenethylpiperidin-4-yl)-N-(1H-1,2,3-triazol-4-yl)propionamide

Over a suspension of N-(1-(4-methoxybenzyl)-1H-1,2,3-triazol-4-yl)-N-(1-phenethylpiperidin-4-yl)propionamide (Ex 25, 300 mg, 0.67 mmol) in THF (3 mL), TFA (3 mL, 40 mmol) was added and the mixture was stirred at 60° C. overnight. Reaction mixture was quenched with toluene and the solvent was evaporated. The crude product thus obtained was purified with sulfonic resin to give the title compound as yellow solid (220 mg, 100% yield).

HPLC-MS (Method A): Ret 1.22 min; ESI⁺-MS m/z, 328.3 (M+1).

### b) Title Compound

To a suspension of the compound obtained in the previous step (80 mg, 0.244 mmol) in anh. toluene (1 mL), Pd₂(dba)₃ (4 mg, 0.04 mmol), tetramethyl ditert-butyl-XPhos (9 mg, 0.02 mmol) and K₃PO₄ (103 g, 0.5 mmol) were added under nitrogen. 1-Bromo-4-fluorobenzene (43 mg, 0.244 mmol) was added and the reaction mixture was heated at 120° C. overnight. The solvent was evaporated and the residue was dissolved in EtOAc and aqueous sat. NaHCO₃ solution. The aqueous layer was extracted with EtOAc and the combined organic layers were dried over Na₂SO₄, filtered and concentrated to give the title compound as green solid (100 mg, quantitative yield).

HPLC-MS (Method A): Ret, 2.36 min; ESI⁺-MS m/z, 422.4 (M+1).

This method was used for the preparation of example 28 using example 24 as starting material:

| | | | | Ret | MS |
|---|---|---|---|---|---|
| Ex | Structure | Chemical name | Method | (min) | (M + H) |
| 28 | 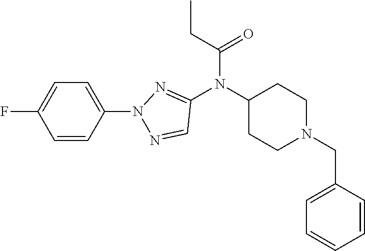 [SMILES: CCC(=O)N(c1cnn(-c2ccc(F)cc2)n1)C1CCN(Cc2ccccc2)CC1] | N-(1- benzylpiperidin-4- yl)-N-(2-(4- fluorophenyl)-2H- 1,2,3-triazol-4- yl)propionamide | A | 2.43 | 408.3 |

### Example 29. N-(1-Benzylpiperidin-4-yl)-N-(1-phenyl-1H-1,2,4-triazol-3-yl)propionamide

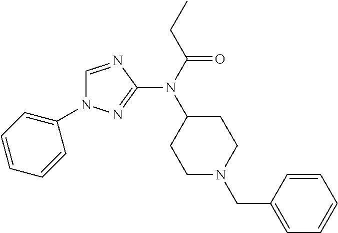 [SMILES: CCC(=O)N(c1ncn(-c2ccccc2)n1)C1CCN(Cc2ccccc2)CC1]

### a) Amino-(2-phenylhydrazono)-methanesulfonic Acid

A mixture of 1-phenyl-3-thiosemicarbazide (1.00 g, 6.0 mmol, 1 eq), Na₂MoO₄.2H₂O (72 mg, 0.3 mmol, 0.05 eq) and NaCl (140 mg, 2.4 mmol, 0.4 eq) in H₂O (3 mL) was cooled to 0° C. A 33% solution of hydrogen peroxide (3.12 mL, 30.3 mmol, 5.05 eq) was added dropwise to the cooled suspension. Once the addition was complete, the mixture was stirred at 0° C. for 1 h. Then it was warmed to room temperature and stirred for 1.5 h. The suspension was filtered and the solid was washed with a small portion of cold brine to give the title compound as a pale brown solid (1.287 g, quant), which was used in the next step without further purification.

HPLC-MS (Method D): Ret, 1.2 min; ESI⁺-MS m/z, 216.2 (M+1).

### b) 1-Benzyl-N-(1-phenyl-1H-1,2,4-triazol-3-yl)-piperidin-4-amine

A mixture of the compound obtained in step a (500 mg, 2.32 mmol, 1.2 eq), 4-amino-1-benzylpiperidine (397 μL, 1.95 mmol, 1 eq) and pyridine (360 μL, 4.45 mmol, 2.3 eq) in anhydrous ACN (4 mL) was heated at 120° C. for 10 min under microwave irradiation. The reaction mixture was then concentrated. The residue was diluted with ethyl orthoformate (4 mL) and EtOH (5 mL) and it was heated at 140° C. for 1 h under microwave irradiation. Afterwards, the solvent was removed and the resulting mixture was dissolved in EtOAc and washed with a saturated solution of NaHCO₃ and brine. The organic layer was dried over anhydrous MgSO₄, filtered and concentrated. The residue was purified by flash chromatography, using mixtures of EtOAc/hex of increasing polarity to provide the title compound as a brown solid (461 mg, 60%).

HPLC-MS (Method D): Ret, 2.4 min; ESI⁺-MS m/z, 334.2 (M+1).

### c) Title Compound

A solution of the compound obtained in step b (100 mg, 0.30 mmol, 1 eq) in excess of propionic anhydride (3 mL) was heated under microwave irradiation at 140° C. for 1.5 h. The excess of propionic anhydride was evaporated under vacuum and the residue was dissolved in EtOAc and washed successively with brine, aqueous 2 N NaOH solution and brine. The organic layer was dried over anhydrous MgSO₄ and the solvent was removed in vacuo. The residue was purified by flash chromatography (EtOAc) to obtain the title compound as a brown gummy solid (55 mg, 51%).

HPLC-MS (Method D): Ret, 2.6 min; ESI⁺-MS m/z, 390.3 (M+1).

This method was used for the preparation of example 30 using example 24 as starting material:

| | | | | Ret | MS |
|---|---|---|---|---|---|
| Ex | Structure | Chemical name | Method | (min) | (M + H) |
| 30 | 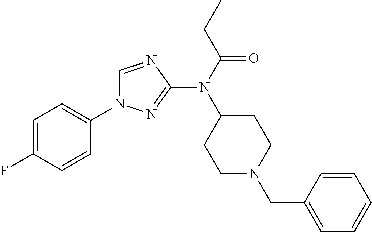 [SMILES: CCC(=O)N(c1ncn(-c2ccc(F)cc2)n1)C1CCN(Cc2ccccc2)CC1] | N-(1- benzylpiperidin-4- yl)-N-(1-(4- fluorophenyl)-1H- 1,2,3-triazol-3- yl)propionamide | D | 2.70 | 408.3 |

### Table of Examples with Binding to the μ-Opioid Receptor and the σ₁-Receptor

### Biological Activity

### Pharmacological Study

### Human σ₁ Receptor Radioligand Assay

To investigate binding properties of test compounds to human σ₁ receptor, transfected HEK-293 membranes and [³H](+)-pentazocine (Perkin Elmer, NET-1056), as the radioligand, were used. The assay was carried out with 7 μg of membrane suspension, 5 nM of [³H](+)-pentazocine in either absence or presence of either buffer or 10 μM Haloperidol for total and non-specific binding, respectively. Binding buffer contained Tris-HCl 50 mM at pH 8. Plates were incubated at 37° C. for 120 minutes. After the incubation period, the reaction mix was then transferred to MultiScreen HTS, FC plates (Millipore), filtered and plates were washed 3 times with ice-cold 10 mM Tris-HCL (pH7.4). Filters were dried and counted at approximately 40% efficiency in a MicroBeta scintillation counter (Perkin-Elmer) using EcoScint liquid scintillation cocktail

### Human μ-Opioid Receptor Radioligand Assay

To investigate binding properties of test compounds to human μ-opioid receptor, transfected CHO-K1 cell membranes and [³H]-DAMGO (Perkin Elmer, ES-542-C), as the radioligand, were used. The assay was carried out with 20 μg of membrane suspension, 1 nM of [³H]-DAMGO in either absence or presence of either buffer or 10 μM Naloxone for total and non-specific binding, respectively. Binding buffer contained Tris-HCl 50 mM, MgCl2 5 mM at pH 7.4. Plates were incubated at 27° C. for 60 minutes. After the incubation period, the reaction mix was then transferred to MultiScreen HTS, FC plates (Millipore), filtered and plates were washed 3 times with ice-cold 10 mM Tris-HCL (pH 7.4). Filters were dried and counted at approximately 40% efficiency in a MicroBeta scintillation counter (Perkin-Elmer) using EcoScint liquid scintillation cocktail.

### Results:

As this invention is aimed at providing a compound or a chemically related series of compounds which act as dual ligands of the σ₁ receptor and the μ-opioid receptor it is a very preferred embodiment in which the compounds are selected which act as dual ligands of the σ₁ receptor and the μ-opioid receptor and especially compounds which have a binding expressed as Kᵢ which is preferably <1000 nM for both receptors, more preferably <500 nM, even more preferably <100 nM.

The following scale as been adopted for representing the binding to the the σ₁ receptor and the μ-opioid receptor expressed as Kᵢ:
+ Both Kᵢ-μ and Kᵢ-σ₁>=500 nM
++ One Kᵢ<500 nM while the other Kᵢ is >=500 nM
+++ Both Kᵢ-μ and Kᵢ-σ₁<500 nM
++++ Both Kᵢ-μ and Kᵢ-σ₁<100 nM

All compounds prepared in the present application exhibit binding to the σ₁ receptor and the μ-opioid receptor, in particular the following binding results are shown:

| | μ and σ1 |
|---|---|
| | dual |
| EX | binding |
| 1 | ++ |
| 2 | +++ |
| 3 | + |
| 4 | +++ |
| 5 | ++ |
| 6 | +++ |
| 7 | ++++ |
| 8 | ++ |
| 9 | +++ |
| 10 | ++ |
| 11 | ++ |
| 12 | ++ |
| 13 | + |
| 14 | + |
| 15 | + |
| 16 | + |
| 18 | + |
| 19 | + |
| 20 | ++ |
| 21 | ++ |
| 22 | ++ |
| 23 | +++ |
| 26 | ++ |
| 27 | ++ |
| 28 | +++ |
| 29 | ++ |
| 30 | ++ |

1-12. (canceled)

13. A compound of Formula (I): 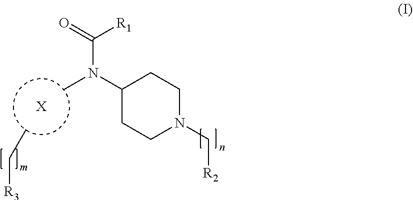
wherein
m is 0, 1, 2, 3, 4 or 5;
n is 1, 2, 3, 4 or 5;
X is a group selected from the group consisting of pyrimidine, pyrazine, oxadiazole, thiazole, thiadiazole, triazole and indazole;
R₁ is selected from the group consisting of substituted or unsubstituted C₁₋₆ alkyl, substituted or unsubstituted C₂₋₆ alkenyl, and substituted or unsubstituted C₂₋₆ alkynyl;
wherein the alkyl, alkenyl or alkynyl in R₁, if substituted, is substituted with one or more substituent/s selected from the group consisting of —OR₄, —C(O)R₄, halogen, —CN, C₁₋₄ haloalkyl, C₁₋₄ haloalkoxy and —NR₄R_{4′};
wherein R₄ is selected from the group consisting of hydrogen, unsubstituted C₁₋₆ alkyl, unsubstituted C₂₋₆ alkenyl and unsubstituted C₂₋₆ alkynyl;
and R_{4′} is selected from the group consisting of hydrogen, unsubstituted C₁₋₆ alkyl, unsubstituted C₂₋₆ alkenyl, unsubstituted C₂₋₆ alkynyl and -Boc;
R₂ is selected from the group consisting of substituted or unsubstituted aryl and substituted or unsubstituted heterocyclyl;
wherein the aryl or heterocyclyl in R₂, if substituted, is substituted with one or more substituent/s selected from the group consisting of halogen, —R₅, —OR₅, —NO₂, —NR₅R_{5′″}, NR₅C(O)R_{5′}, —NR₅S(O)₂R_{5′}, —S(O)₂NR₅R_{5′}, —NR₅C(O)NR_{5′}R_{5″}, —SR₅, —S(O)R₅, S(O)₂R₅, —CN, C₁₋₄ haloalkyl, C₁₋₄ haloalkoxy, —C(O)OR₅, —C(O)NR₅R_{5′}, —OCH₂CH₂OH, —NR₅S(O)₂NR_{5′}R_{5″} and —C(CH₃)₂OR₅;
and wherein the non-aromatic heterocyclyl in R₂, if substituted, may also be spirosubstituted or substituted with ═O;
wherein R₅, R_{5′} and R_{5″} are independently selected from the group consisting of hydrogen, unsubstituted C₁₋₆ alkyl, unsubstituted C₂₋₆ alkenyl and unsubstituted C₂₋₆ alkynyl;
and wherein R_{5′″} is selected from the group consisting of hydrogen, unsubstituted C₁₋₆ alkyl, unsubstituted C₂₋₆ alkenyl, unsubstituted C₂₋₆ alkynyl and -Boc;
R₃ is selected from the group consisting of substituted or unsubstituted aryl and substituted or unsubstituted heterocyclyl;
wherein the aryl or heterocyclyl in R₃, if substituted, is substituted with one or more substituent/s selected from the group consisting of halogen, —R₆, —OR₆, —NO₂, —NR₆R_{6′″}, NR₆C(O)R_{6′}, —NR₆S(O)₂R_{6′}, —S(O)₂NR₆R_{6′}, —NR₆C(O)NR_{6′}R_{6″}, —SR₆, —S(O)R₆, S(O)₂R₆, —CN, C₁₋₄ haloalkyl, C₁₋₄ haloalkoxy, —C(O)OR₆, —C(O)NR₆R_{6′}, —OCH₂CH₂OH, —NR₆S(O)₂NR_{6′}R_{6″} and —C(CH₃)₂OR₆;
and wherein the non-aromatic heterocyclyl in R₃, if substituted, may also be spirosubstituted or substituted with ═O;
wherein R₆, R_{6′} and R_{6″} are independently selected from the group consisting of hydrogen, unsubstituted C₁₋₆ alkyl, unsubstituted C₂₋₆ alkenyl and unsubstituted C₂₋₆ alkynyl;
and wherein R_{6′″} is selected from the group consisting of hydrogen, unsubstituted C₁₋₆ alkyl, unsubstituted C₂₋₆ alkenyl, unsubstituted C₂₋₆ alkynyl and -Boc;
optionally as a stereoisomer, including enantiomers and diastereomers, a racemate or a mixture of at least two of stereoisomers, including enantiomers and/or diastereomers, in any mixing ratio, or a corresponding salt thereof, or a corresponding solvate thereof.

14. The compound according to claim 13, wherein R₁ is selected from the group consisting of substituted or unsubstituted C₁₋₆ alkyl, substituted or unsubstituted C₂₋₆ alkenyl, and substituted or unsubstituted C₂₋₆ alkynyl.

15. The compound according to claim 13, wherein R₁, is substituted or unsubstituted C₁₋₆ alkyl.

16. The compound according to claim 13, wherein R₁, is substituted or unsubstituted ethyl or substituted or unsubstituted —CH(CH₃)C(O)-ethyl.

17. The compound according claim 13, wherein R₂ is selected from the group consisting of substituted or unsubstituted aryl and substituted or unsubstituted heterocyclyl.

18. The compound according to claim 13, wherein R₂ is substituted or unsubstituted aryl.

19. The compound according to claim 13, wherein R₂ is substituted or unsubstituted phenyl.

20. The compound according to claim 13, wherein R₂ is unsubstituted phenyl.

21. The compound according to claim 13, wherein R₃ is selected from the group consisting of substituted or unsubstituted aryl and substituted or unsubstituted heterocyclyl.

22. The compound according to claim 13, wherein R₃ is substituted or unsubstituted aryl.

23. The compound according to claim 13, wherein R₃ is substituted or unsubstituted phenyl.

24. The compound according to claim 13, wherein the compound of Formula (I) is a compound of Formula (I⁵′) 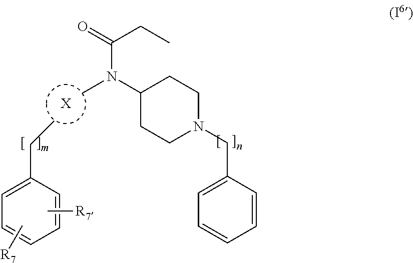
wherein R₇ and R_{7′} are independently selected from the group consisting of hydrogen, halogen, —R₆, —OR₆, —NO₂, —NR₆R_{6′″}, NR₆C(O)R_{6′}, —NR₆S(O)₂R_{6′}, —S(O)₂NR₆R_{6′}, —NR₆C(O)NR_{6′}R_{6″}, —SR₆, —S(O)R₆, S(O)₂R₆, —CN, haloalkyl, haloalkoxy, —C(O)OR₆, —C(O)NR₆R_{6′}, —OCH₂CH₂OH, —NR₆S(O)₂NR_{6′}R_{6″} and —C(CH₃)₂OR₆;
and R₆, R_{6′}, R_{6″}, R_{6′″}, X, m and n are as defined in claim 13.

25. The compound according to claim 24, wherein R₇ and R_{7′} are independently selected from the group consisting of hydrogen, halogen, and —OR₆.

26. The compound according to claim 24, wherein R₇ and R_{7′} are independently selected from the group consisting of hydrogen, fluorine, chlorine, —OH and substituted or unsubstituted —O-methyl.

27. The compound according to claim 13, wherein the compound of Formula (I) is a compound of Formula (I⁶′) 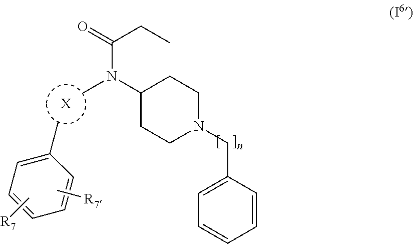
wherein R₇ and R_{7′} are independently selected from hydrogen, halogen, —R₆, —OR₆, —NO₂, —NR₆R_{6′″}, NR₆C(O)R_{6′}, —NR₆S(O)₂R_{6′}, —S(O)₂NR₆R_{6′}, —NR₆C(O)NR_{6′}R_{6″}, —SR₆, —S(O)R₆, S(O)₂R₆, —CN, haloalkyl, haloalkoxy, —C(O)OR₆, —C(O)NR₆R_{6′}, —OCH₂CH₂OH, —NR₆S(O)₂NR_{6′}R_{6″} and —C(CH₃)₂OR₆;
and R₆, R_{6′}, R_{6″}, R_{6′″}, X and n are as defined in claim 1.

28. The compound according to claim 27, wherein R₇ and R_{7′} are independently selected from the group consisting of hydrogen, halogen, and —OR₆.

29. The compound according to claim 27, wherein R₇ and R_{7′} are independently selected from the group consisting of hydrogen, fluorine, chlorine, —OH and —O-methyl.

30. The compound according to claim 13, wherein the compound is selected from:
N-(1-benzylpiperidin-4-yl)-N-(2-(4-fluorophenyl)pyrimidin-5-yl)propionamide,
N-(1-benzylpiperidin-4-yl)-N-(6-(4-fluorophenyl)pyrazin-2-yl)propionamide,
N-(1-benzylpiperidin-4-yl)-N-(5-(4-fluorophenyl)pyrazin-2-yl)propionamide,
N-(1-benzylpiperidin-4-yl)-N-(5-phenyl-1,3,4-oxadiazol-2-yl)propionamide,
N-(1-benzylpiperidin-4-yl)-N-(3-phenyl-1,2,4-thiadiazol-5-yl)propionamide,
N-(1-benzylpiperidin-4-yl)-N-(5-phenyl-1,3,4-thiadiazol-2-yl)propionamide,
N-(1-benzylpiperidin-4-yl)-N-(4-phenylthiazol-2-yl)propionamide,
N-(1-benzylpiperidin-4-yl)-2-methyl-3-oxo-N-(4-phenylthiazol-2-yl)pentanamide,
N-(1-benzylpiperidin-4-yl)-N-(5-phenylthiazol-2-yl)propionamide,
N-(2-(4-fluorophenyl)pyrimidin-5-yl)-N-(1-phenethylpiperidin-4-yl)propionamide,
N-(6-(4-fluorophenyl)pyrazin-2-yl)-N-(1-phenethylpiperidin-4-yl)propionamide,
N-(5-(4-fluorophenyl)pyrazin-2-yl)-N-(1-phenethylpiperidin-4-yl)propionamide,
N-(1-benzylpiperidin-4-yl)-N-(1-phenyl-1H-indazol-3-yl)propionamide,
N-(1-benzylpiperidin-4-yl)-N-(1-(4-chlorophenyl)-1H-indazol-3-yl)propionamide,
N-(1-benzylpiperidin-4-yl)-N-(1-(4-fluorophenyl)-1H-indazol-3-yl)propionamide,
N-(1-benzylpiperidin-4-yl)-N-(1-(3-chloro-4-fluorophenyl)-1H-indazol-3-yl)propionamide,
N-(1-benzylpiperidin-4-yl)-N-(1-(4-methoxyphenyl)-1H-indazol-3-yl)propionamide,
N-(1-benzylpiperidin-4-yl)-N-(1-(4-hydroxyphenyl)-1H-indazol-3-yl)propionamide,
N-(1-benzylpiperidin-4-yl)-N-(1-(4-fluorophenyl)-1H-1,2,3-triazol-4-yl)propionamide,
N-(1-(4-fluorophenyl)-1H-1,2,3-triazol-4-yl)-N-(1-phenethylpiperidin-4-yl)propionamide,
N-(1-(3,4-dichlorobenzyl)-1H-1,2,3-triazol-4-yl)-N-(1-phenethylpiperidin-4-yl)propionamide,
N-(1-(4-chloro-3-fluorophenyl)-1H-1,2,3-triazol-4-yl)-N-(1-phenethylpiperidin-4-yl)propionamide,
N-(1-(3,4-dichlorobenzyl)-1H-1,2,3-triazol-4-yl)-N-(1-phenethylpiperidin-4-yl)acetamide,
N-(1-benzylpiperidin-4-yl)-N-(1-(4-methoxybenzyl)-1H-1,2,3-triazol-4-yl)propionamide,
N-(1-(4-methoxybenzyl)-1H-1,2,3-triazol-4-yl)-N-(1-phenethylpiperidin-4-yl)propionamide,
N-(1-(4-fluorophenyl)-1H-1,2,3-triazol-5-yl)-N-(1-phenethylpiperidin-4-yl)propionamide,
N-(2-(4-fluorophenyl)-2H-1,2,3-triazol-4-yl)-N-(1-phenethylpiperidin-4-yl)propionamide,
N-(1-benzylpiperidin-4-yl)-N-(2-(4-fluorophenyl)-2H-1,2,3-triazol-4-yl)propionamide,
N-(1-benzylpiperidin-4-yl)-N-(1-phenyl-1H-1,2,3-triazol-4-yl)propionamide, and
N-(1-benzylpiperidin-4-yl)-N-(1-(4-fluorophenyl)-1H-1,2,4-triazol-3-yl)propionamide
optionally as a stereoisomer, including enantiomers and diastereomers, a racemate or a mixture of at least two stereoisomers, including enantiomers and/or diastereomers, in any mixing ratio, or a corresponding salt thereof, or a corresponding solvate thereof.

31. A process for the preparation of compound the compound of Formula (I) according to claim 13 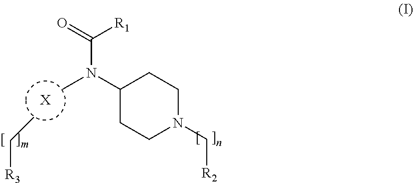
which process comprises acylation of a compound of formula IVb 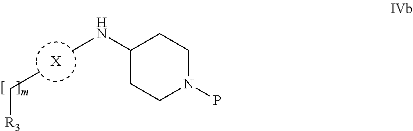
wherein P represents the following moiety 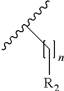
with a compound of formula VIa or a compound formula VIb 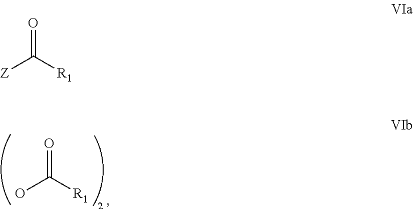
wherein Z represents a suitable leaving group, including a halogen or an ethoxy or methoxy group,
or
which process comprises alkylation of a compound of Formula VIII, 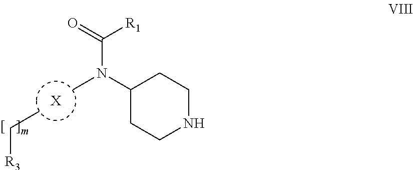
with a compound of formula IXa, 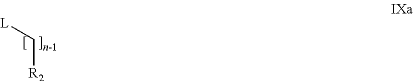
wherein L is a suitable leaving group, including a halogen, mesylate, tosylate or triflate,
or
which process comprises a reductive amination reaction between a compound of formula VIII, 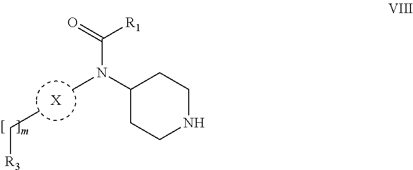
and a compound of formula IXb, 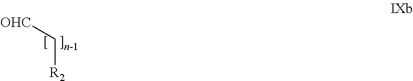
Or,
in the case wherein formula I 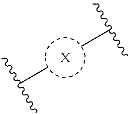
is triazole,
which process comprises reaction of the compound XIb 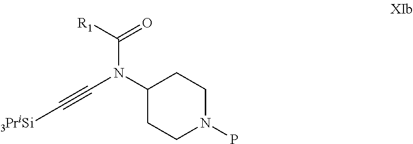
wherein P represents a moiety Y 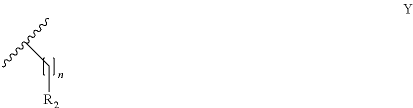
with an azide derivative of formula XII 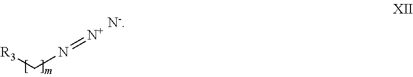
wherein X, R₁, R₂, R₃, n and m, unless defined otherwise, are as defined in claim 13.

32. A process for the preparation of the compound of Formula (I) according to claim 13, employing a compound of Formula IIa, IIb, IIIa, IIIb, IVa, IVb, Va, Vb, VIa, VIb, VII, VIII, IXa, IXb, Xa, Xb, XIa, XIb or XII, 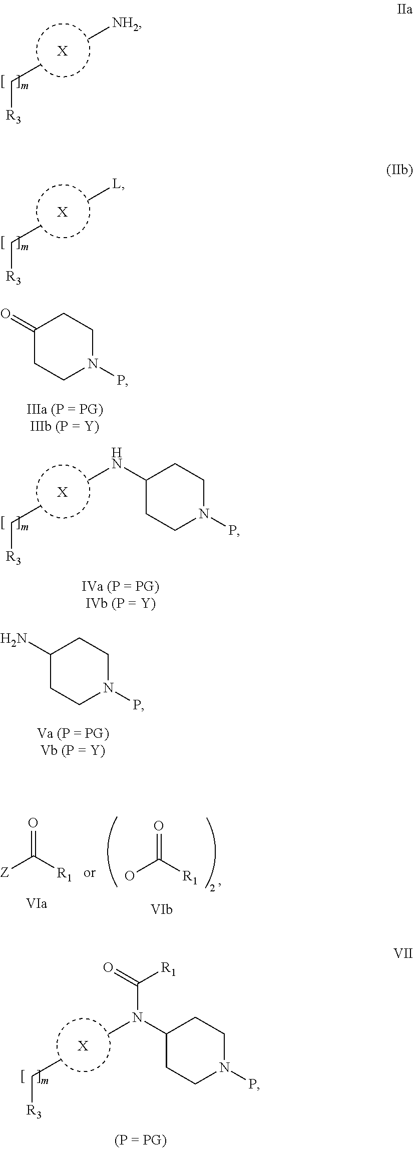 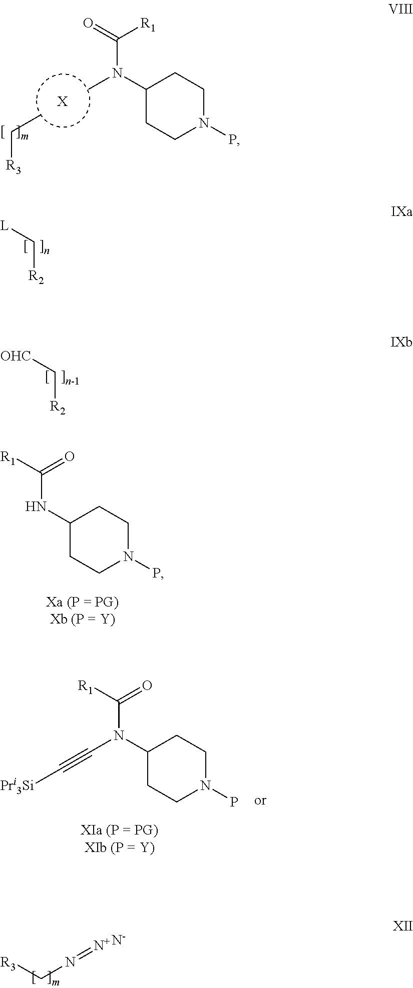
wherein L and Z represents a suitable leaving group, including a halogen, P represents a protective group (PG) or the moiety Y 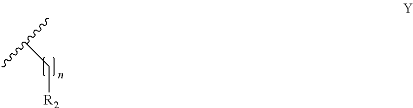
and X, R₁, R₂, R₃, n and m are as defined in claim 13.

33. A pharmaceutical composition which comprises the compound according to claim 13, or a pharmaceutically acceptable salt thereof, and a pharmaceutically acceptable carrier, adjuvant or vehicle.

34. A method of treating pain in a subject in need thereof, comprising administration of an effective amount of the compound according to claim 13.

35. The method according to claim 34, wherein the pain is selected from the group consisting of medium to severe pain, visceral pain, chronic pain, cancer pain, migraine, inflammatory pain, acute pain or neuropathic pain, allodynia and hyperalgesia.
